(19) United States
(12) Patent Application Publication
(10) Pub. No.: US 2026/0297679 A1
(43) Pub. Date: Oct. 1, 2026

(54) ASSAYS FOR MONITORING INHIBITION OF RNA HELICASE DHX9

(71) Applicant: ACCENT THERAPEUTICS, INC., Lexington, MA (US)

(72) Inventors: Jennifer Castro, Billerica, MA (US); Scott Ribich, Lexington, MA (US); David Patrick Brennan, Wakefield, MA (US)

(73) Assignee: ACCENT THERAPEUTICS, INC., Lexington, MA (US)

(21) Appl. No.: 19/475,686

(22) PCT Filed: Apr. 15, 2024
(86) PCT No.: PCT/US2024/024695

§ 371 Date: Oct. 15, 2025

Related U.S. Application Data
(60) Provisional application No. 63/459,632, filed on Apr. 15, 2023.

(51) Int. Cl. C12Q 1/6886

(52) U.S. Cl. CPC C12Q 1/6886; C12Q 2600/106; C12Q 2600/136; C12Q 2600/158; C12Q 2600/178

(57) ABSTRACT

The present disclosure provides methods of measuring the activity of a DHX9 inhibitor in cells or in a subject, the methods comprising measuring the level of an Alu-mediated circular RNA induced by the DHX9 inhibitor.

### REFERENCES TO RELATED APPLICATIONS

This application claims priority to U.S. Provisional Application No. 63/459,632, filed on Apr. 15, 2023, the entire content of which is incorporated herein by reference

### SEQUENCE LISTING

The instant application contains a Sequence Listing XML which has been submitted electronically in XML format and is hereby incorporated by reference in its entirety. Said XML copy, created on Apr. 15, 2024, is named 130090-01520.xml and is 51,303 bytes in size.

### BACKGROUND OF THE INVENTION

Genomic instability is a characteristic of most cancers. In hereditary cancers, genomic instability results from mutations in DNA repair genes and drives cancer development.

Interfering with players that maintain genome stability or manage replication stress, such as RNA helicases, is an evolving an approach to specifically target cancer with genomic instability.

DEAD/H box RNA helicases are an interesting group of proteins serving as potential translational targets in cancer. These proteins belong to superfamily 2, the largest group of eukaryotic RNA helicases, are named after a conserved amino sequence (Asp-Glu-Ala-Asp/His) and have the ability to unwind and restructure RNA molecules with complex secondary structures in an ATPase dependent fashion. They remodel complex RNA structures like hairpins and mRNP complexes and have been reported to play a pivotal role in virtually all steps of mRNA processing and translation. Interestingly, cancer cells seem to rely heavily on RNA helicases to meet not only the increased general protein synthesis demand, but also for translation of specific pro-oncogenic mRNAs to enhance survival.

DHX9, also known as RNA Helicase A (RHA) or Nuclear DNA Helicase II (NDH II), is a DEAH-box RNA helicase. Due to its regulatory role in processes such as transcription and maintenance of genomic stability, DHX9 has been shown to be a key regulator in a variety of cancer types (Gulliver et al., 2020, Future Science OA (2), FS0650). Specifically, microsatellite instable cancers, such as Microsatellite Instable (MSI) colorectal cancer, and tumors with defective Mismatch Repair (MMR) exhibit a strong dependence on DHX9. Thus, DHX9 is a promising target for treating certain types of cancers.

With the development of DHX9 inhibitors as potential therapeutic agents for treating certain cancers, there is a need in the art for non-invasive biomarkers for their clinical applications.

### SUMMARY OF THE INVENTION

In one aspect, the present disclosure provides a method of measuring activity of a DHX9 inhibitor in a cell, comprising: contacting a cell with the DHX9 inhibitor; and measuring the level of an Alu-mediated circular RNA (Alu-circRNA) in the cell.

In some embodiments, the cell is a cancer cell. In some embodiments, the cell is a normal cell. In some embodiments, the cell is a non-cancerous cell. In some embodiments, the cell is a peripheral blood mononuclear cell (PBMC).

In some embodiments, the contacting is for a sufficient time for the Alu-circRNA to be produced.

In some embodiments, the level of Alu-circRNA is correlated to inhibition of a DHX9 activity by the DHX9 inhibitor in the cell.

In another aspect, the present disclosure provides a method of measuring activity of a DHX9 inhibitor in a subject, comprising: administering a DHX9 inhibitor to a subject; and measuring the level of an Alu-mediated circular RNA (Alu-circRNA) in a biological sample from the subject.

In another aspect, the present disclosure provides a method of measuring activity of a DHX9 inhibitor in a subject, comprising: measuring the level of Alu-mediated circular RNA (Alu-circRNA) in a biological sample from a subject, wherein the subject is being treated with a DHX9 inhibitor.

In some embodiments, the level of Alu-circRNA is correlated to inhibition of DHX9 activity by the DHX9 inhibitor in the subject. In some embodiments, the level of Alu-circRNA is correlated to inhibition of DHX9 ATPase activity or resolvase activity. In some embodiments, the level of Alu-circRNA is correlated to inhibition of proliferation of MSI cancer cells in the subject.

In some embodiments, the biological sample is selected from the group consisting of blood, interstitial fluid, lymph fluid, organ tissue, biopsy tissue, and skin tissue.

In some embodiments, the biological sample is blood. In some embodiments, the method further comprises isolating cells from the blood sample, and measuring the level of the Alu-circRNA in the isolated cells.

In some embodiments, the biological sample comprises peripheral blood mononuclear cells (PBMCs). In some embodiments, the method further comprises isolating PBMCs from a blood sample, and measuring the level of the Alu-circRNA in the PBMCs. In some embodiments, the level of Alu-circRNA does not correlate with inhibition of proliferation of PBMCs of the subject.

In some embodiments, the Alu-circRNA is selected from the group consisting of cirRNA of BRIP1, AKR1A1, BMPR2, CALCOCO2, CLNSIA, DKC1, FAM124A, FBN1, FCHO2, GLS, GON4L, GPR125, NEIL3, PIK3C3, PNN, POMT1, RBM23, SKIL, SLK, SMA, SMG1, SMYD4, TAF2, TFAP4, TMEM41B, USP25, VPS13D, WDR59, and XPR1.

In some embodiments, the level of the Alu-circRNA is measured by a technique selected from northernblot, reverse transcription-quantitative polymerase chain reaction (RT-qPCR), microarray, droplet digital PCR, next-generation sequence (NGS) RNA sequencing, RNA in situ hybridization (RNA-ISH), in situ hybridization-immunohistochemistry (ISH-IHC), isothermal exponential amplification, and rolling cycle amplification.

In some embodiments, the method further comprises obtaining the biological sample from the subject. In some embodiments, the biological sample is obtained from the subject between 0.5-7 days following administration of the DHX9 inhibitor. In some embodiments, the method comprises measuring the level of the Alu-circRNA in biological samples obtained from the subject at more than one time point.

In some embodiments, the subject has a cancer. In some embodiments, the cancer is a microsatellite instability (MSI) cancer. In some embodiments, the cancer is selected from the group consisting of colorectal, endometrial, ovarian, gastric, hematopoietic, breast, brain, skin, lung, blood, prostate, head and neck, pancreatic, bladder, bone, soft tissue, kidney, liver, and Ewing's sarcoma.

In some embodiments, the method further comprises making a determination on an appropriate therapeutic dose of the DHX9 inhibitor based on a set of data including the level of Alu-circRNA measured.

In some embodiments, the DHX9 inhibitor is selected from a small molecule, an antisense oligonucleotide, small interfering RNA (siRNA), a small hairpin RNA (shRNA), a microRNA (miRNA), or a piwiRNA (piRNA).

In some embodiments, the DHX9 inhibitor is a small molecule. In some embodiments, the DHX9 inhibitor is selected from the inhibitors in Table 1.

In some embodiments, the subject is a human.

In yet another aspect, the present disclosure provides a method of monitoring treatment of a subject with a DHX9 inhibitor, comprising: administering a DHX9 inhibitor to the subject; and obtaining information as to the level of Alu-mediated circular RNA (Alu-circRNA) in a biological sample obtained from the subject after administration of the DHX9 inhibitor.

In some embodiments, the level of Alu-circRNA is correlated to inhibition of DHX9 activity by the DHX9 inhibitor in the subject. In some embodiments, the level of Alu-circRNA is correlated to inhibition of DHX9 ATPase activity or resolvase activity. In some embodiments, the level of Alu-circRNA is correlated to inhibition of proliferation of MSI cancer cells in the subject.

In some embodiments, the biological sample is selected from the group consisting of blood, interstitial fluid, lymph fluid, organ tissue, biopsy tissue, and skin tissue.

In some embodiments, the biological sample is blood. In some embodiments, the method further comprises isolating cells from the blood sample, and measuring the level of the Alu-circRNA in the isolated cells.

In some embodiments, the biological sample comprises peripheral blood mononuclear cells (PBMCs). In some embodiments, the method further comprises isolating PBMCs from a blood sample, and measuring the level of the Alu-circRNA in the PBMCs. In some embodiments, the level of Alu-circRNA does not correlate with inhibition of proliferation of PBMCs of the subject.

In some embodiments, the Alu-circRNA is selected from the group consisting of cirRNA of BRIP1, AKR1A1, BMPR2, CALCOCO2, CLNSIA, DKC1, FAM124A, FBN1, FCHO2, GLS, GON4L, GPR125, NEIL3, PIK3C3, PNN, POMT1, RBM23, SKIL, SLK, SMA, SMG1, SMYD4, TAF2, TFAP4, TMEM41B, USP25, VPS13D, WDR59, and XPR1.

In some embodiments, the level of the Alu-circRNA is measured by a technique selected from northernblot, reverse transcription-quantitative polymerase chain reaction (RT-qPCR), microarray, droplet digital PCR, next-generation sequence (NGS) RNA sequencing, RNA in situ hybridization (RNA-ISH), in situ hybridization-immunohistochemistry (ISH-IHC), isothermal exponential amplification, and rolling cycle amplification.

In some embodiments, the method further comprises obtaining the biological sample from the subject. In some embodiments, the biological sample is obtained from the subject between 0.5-7 days following administration of the DHX9 inhibitor. In some embodiments, the method comprises measuring the level of the Alu-circRNA in biological samples obtained from the subject at more than one time point.

In some embodiments, the subject has a cancer. In some embodiments, the cancer is a microsatellite instability (MSI) cancer. In some embodiments, the cancer is selected from the group consisting of colorectal, endometrial, ovarian, gastric, hematopoietic, breast, brain, skin, lung, blood, prostate, head and neck, pancreatic, bladder, bone, soft tissue, kidney, liver, and Ewing's sarcoma.

In some embodiments, the method further comprises making a determination on an appropriate therapeutic dose of the DHX9 inhibitor based on a set of data including the level of Alu-circRNA measured.

In some embodiments, the DHX9 inhibitor is selected from a small molecule, an antisense oligonucleotide, small interfering RNA (siRNA), a small hairpin RNA (shRNA), a microRNA (miRNA), or a piwiRNA (piRNA).

In some embodiments, the DHX9 inhibitor is a small molecule. In some embodiments, the DHX9 inhibitor is selected from the inhibitors in Table 1.

In some embodiments, the subject is a human.

### BRIEF DESCRIPTION OF THE DRAWINGS

FIG. 1: Cells were treated with DHX9 compound 7 for 3 days and probed for multiple Alu-mediated circRNAs, a non-Alu mediated circRNA, as well as each gene's respective linear RNA form. Upon DHX9 inhibition, the Alu-mediated circRNAs, BRIP1, AKR1A1 and DKC1, were regulated and induced.

FIGS. 2A-2B show that cellular target engagement by DHX9 inhibitors correlates with DHX9 biochemical activity and anti-proliferative activity of DHX9 inhibitors in certain MSI cancers. FIG. 2A shows DHX9 ATPase biochemical activity for each DHX9 inhibitor, reported as inflection points (IP) or EC50, plotted against its corresponding circBRIP1 EC50, which is EC50 of circBRIP1 induced in HCT116 CRC-MSI cells measured by cellular DHX9 target engagement assays. FIG. 2B shows anti-proliferative activity of each DHX9 inhibitor in LS411N CRC-MSI cells, measured as IC50, plotted against its corresponding circBRIP1 EC50, measured by cellular DHX9 target engagement assays.

FIGS. 3A-3D: DHX9 compounds (1 to 4) were dosed in vivo as described in Example 3. Tumor samples were taken at the end of the study 1 hr post last dose and analyzed for human linear BRIP1 and/or human circBRIP1. Dose dependent induction of circBRIP1 was observed and where applicable, no change in linear BRIP1 was seen.

FIGS. 4A-4C: Mice with human xenograft tumors were dosed with DHX9 inhibitor Compound 1 at 30, 100, 200 and 300 mg/kg orally with a 12 hour schedule as described in Example 4. After 21 days of treatment and 12 hours post last dose of compound tumor volume was recorded, tumors were then harvested and processed for circBRIP1 expression.

FIG. 4A shows dose-dependent induction of circBRIP1 in LS411N tumors. FIG. 4B shows individual tumor volumes plotted against each tumor's corresponding circBRIP1 pharmacodynamic reading in a correlation plot. FIG. 4C shows individual tumor drug exposure of Compound 1 plotted against each tumor's corresponding circBRIP1 pharmacodynamic reading and fit to four parameter non-linear regression.

FIGS. 5A-5B: DHX9 compounds were dosed by oral gavage (PO) in vivo as described in Example 5. Whole blood samples were taken at the end of the study 1 hr post last dose, PBMC were isolated and analyzed for mouse linear BRIP1 and/or mouse circBRIP1. Dose dependent induction of circBRIP1 was observed and where applicable, no change in linear BRIP1 was seen.

FIG. 6: DHX9 compounds were dosed oral gavage (PO) in vivo as described in Example 5. Whole blood samples were taken at the end of the study 24 hr post last dose, PBMC were isolated and analyzed for mouse linear BRIP1 and/or mouse circBRIP1. Dose dependent induction of circBRIP1 was observed, while no linear BRIP1 above baseline was seen.

FIG. 7A: Colorector cancer cell lines that were either sensitive to DHX9 inhibitor (IC₅₀<1 μM or insensitive to DHX9 inhibitor (IC₅₀>1 μM) were treated with escalating doses of from 0.001 to 1 μM Compound 1, as described in Example 6. circBRIP1 EC₅₀ values were relative to vehicle treated cell. Similar potency of circBRIP1 induction was seen in both sensitive and insensitive cells. FIG. 7B: Several breast, ovarian, lung and colorectal (CRC) cancer cell lines were treated with Compound 1 as described in Example 6. IC50 values for inhibition of proliferative activity by Compound 1 in the cell lines showed no correlation with EC50 values of circBRIP1 induction.

FIG. 8A: circBRIP1 level was elevated in previously-frozen isolated human PBMCs after treatment with Compound 1 for 24 hours. FIG. 8B: Treatment of PBMCs with Compound 1 at concentration of 0.01 μM to 10 μM for 10 days did not impact their proliferation (staustoporine: positive control).

FIGS. 9A-9D: Four fresh human blood samples were obtained and processed as described in Example 8. Results show an induced level of human circBRIP1 in a dose response when treated with DHX9 compound 1 at varying levels per donor. Linear BRIP1 was not significantly changed.

### DETAILED DESCRIPTION OF THE INVENTION

### I. Definitions

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. The term “such as” is used herein to mean, and is used interchangeably, with the phrase “such as but not limited to.”

As used herein, the term “a,” “an,” “the” and similar terms used in the context of the present disclosure (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the disclosure and does not pose a limitation on the scope of the disclosure otherwise claimed.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “Alu element” refers to a short stretch of DNA (about 300 nucleotides in length), originally characterized by the action of the Alu restriction endonuclease and classified as short interspersed nuclear elements (SINEs). Alu elements are the most abundant transposable elements, and can be divided into five subfamilies of related elements based upon key diagnostic nucleotide positions shared by subfamily members.

Typically, an Alu element is composed of two non-identical units or arms joined in the middle by an adenosine (A)-rich linker. Typically, each Alu repeat also ends with an A-rich tail and is flanked by short direct repeat sequences. Many Alu elements have been annotated and characterized in the art (see, e.g., Dagan et al. Alu Gene: a database of Alu elements incorporated within protein-coding genes, Nucleic Acids Research, 32(1), 2004, Pages D489-D492; Hormozdiari et al., Alu repeat discovery and characterization within human genomes Genome Res., 2011, 21: 840-849; the entire contents of each of which are incorporated herein by reference) As used herein, the term “biomarker”, used interchangeably with the term “marker”, is understood to mean a measurable characteristic that reflects in a quantitative or qualitative manner the physiological state of a cell or an organism. Said another way, biomarkers are characteristics that can be objectively measured and evaluated as indicators of normal processes, pathogenic processes, or pharmacologic responses to a therapeutic intervention.

Examples of biomarkers include, for example, polypeptides, peptides, polypeptide fragments, proteins, antibodies, hormones, polynucleotides, RNA or RNA fragments, microRNA (miRNAs), circular RNA (circRNAs), lipids, polysaccharides, and other bodily metabolites.

In a preferred embodiment, a biomarker is a circRNA, e.g., an Alu-mediated circRNA Preferably, a biomarker of the present invention is modulated (e.g., increased or decreased level) in a cell or a biological sample from a subject or a group of subjects after having received a treatment as compared to a biological sample from a subject or group of subjects that have not received a treatment (e.g., a control). In some embodiments a biomarker of the present invention is modulated (e.g., increased or decreased level) in a biological sample from a subject or a group of subjects after having received a treatment as compared to a biological sample from the subject or group of subjects before receiving the treatment (e.g., a control).

A biomarker may be differentially present at any level, but is generally present at a level that is increased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased relative to normal or control levels by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using either Welch's T-test or Wilcoxon's rank-sum Test).

As used herein, the term “biopsy” or “biopsy tissue” refers to a sample of tissue (e.g., prostate tissue) that is removed from a subject for the purpose of determining if the sample contains cancerous tissue. The biopsy tissue is then examined (e.g., by microscopy) for the presence or absence of cancer.

As used herein, the term “cancer” has the meaning normally accepted in the art. The term can broadly refer to abnormal cell growth.

As used herein, the term “circular RNA,” abbreviated as “circRNA,” refers to noncoding RNA characterized by a covalently closed cyclic structure lacking poly-adenylated tails. CircRNAs are mainly synthesized by the transcription of protein coding genes with RNA polymerase II (Pol II); but unlike linear RNAs, they are not produced by canonical mode of RNA splicing. CircRNA molecules are circularized by joining the 3′ and 5′ ends together with unique back-splicing (see, e.g., Wilusz, Repetitive elements regulate circular RNA biogenesis, Mob Genet Elements, 2015, 5(3):39-45; incorporated herein by reference). CircRNAs are commonly named according to their parental genes or specific functions, e.g., circular RNA derived from the BRIP1 gene is called “circBRIP1.” Several circRNA databases have been constructed to enable organization of discovered and identified circRNAs. A serial number is given to every detected back-spliced junction site. Databases like circBase (circbase.org) and CircNet (circnet. mbc.nctu.edu.tw) provide tissue-specific circRNA expression profiles as well as circRNA-miRNA-gene regulatory networks. Circ2Traits (gyanxet-beta.com/circdb) also allows user to search circRNAs by multiple diseases. See Zhang et al. (Circular RNAs: Promising Biomarkers for Human Diseases, EBioMedicine 34 (2018) 267-274), the entire contents of which are incorporated herein by reference.

The term “Alu-mediated circular RNA”, abbreviated as “Alu-circRNA” herein, refers to circRNA whose formation is associated with Alu elements. Alu elements are enriched in the introns flanking human exons that generate circRNAs; it is estimated that ~90% of circular RNAs appear to have complementary Alu elements in their flanking introns. Disruption of base pairing between intronic repeats by mutating several nucleotides in Alu elements have been shown to prevent circularization of certain RNAs (see, e.g., Wilusz, Repetitive elements regulate circular RNA biogenesis, Mob Genet Elements, 2015, 5(3):39-45; the entire contents of which are incorporated herein by reference). In some embodiments, the formation of the Alu-circRNAs of the instant disclosure are modulated by DHX9.

As used herein, the term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control sample,” as used herein, refers to any relevant comparative sample, including, for example, a sample from a subject prior to treatment or from an earlier assessment time point, or a cell or a population of cells untreated with any agents. A control sample can be a purified sample and/or nucleic acid (e.g., circRNA) provided with a kit. A control sample can be a sample derived from a subject. A control sample can also be synthetically produced. The level of one or more markers (e.g., 1, 2, 3, 4, 5, 6, 7, 8, or 9 or more markers) in a control sample consists of a group of measurements that may be determined, e.g., based on any appropriate statistical measurement, such as, for example, measures of central tendency including average, median, or modal values. Different from a control is preferably statistically significantly different from a control.

The term “control level” refers to an accepted or pre-determined level of a marker in a sample. A control level can be a range of values. Marker levels can be compared to a single control value, to a range of control values, to the upper level of normal, or to the lower level of normal as appropriate for the assay.

In some embodiments, the control level is a standardized control level, such as, for example, a level which is predetermined using an average of the levels of expression of one or more markers from a population of cells or subjects prior to receiving a treatment, e.g., prior to administration of a DHX9 inhibitor. In some embodiments, the control level is a level determined from a sample of a subject collected before the subject receiving a treatment, e.g., prior to administration of a DHX9 inhibitor. In some embodiments, the control level is a level determined from a sample of a subject collected at an earlier time point.

As used herein, “detecting”, “detection”, “determining”, and the like are understood to refer to an assay performed for identification of the presence and/or level of a circRNA (e.g., circBRIP1) and/or an additional one or more specific markers in a sample. The amount of marker detected in the sample can be none or below the level of detection of the assay or method.

As used herein, the term “DHX9”, RNA Helicase A (RHA) or Nuclear DNA Helicase II (NDH II) refers to a DEAH-box RNA helicase which shuttles between nucleus and cytoplasm, and can use all four NTPs to power cycles of directional movement from 3′ to 5′. Functionally, DHX9 can bind to and unwind or resolve dsDNA/RNA, ssDNA/RNA, DNA:RNA hybrids (such as R-loops), circular RNA, and DNA/RNA G quadruplexes. As such, DHX9 has regulatory roles in various RNA and DNA related cellular processes, such as transcription, translation, RNA splicing, editing, RNA transport and processing, microRNA genesis, and maintenance of genomic stability (Pan et al., 2021, Current Protein & Peptide Science (22), 29-40). The NCBI Gene ID for DHX9 is 1660. Human DHX9 nucleotide and amino acid sequences can be found at GenBank Accession No. NM_001357.5 (transcript variant 1) and NR_033302.2 (transcript variant 2). (The GenBank number is incorporated herein by reference in the version available on the filing date of the application to which this application claims priority).

DHX9 possesses ATPase activity, i.e., DHX9 has an ATPase domain that hydrolyzes ATP into ADP. It has recently been shown that DHX9 also possesses resolvase activity, which represses transposases (see Akta et al., DHX9 suppresses RNA processing defects originating from the Alu invasion of the human genome, Nature, 2017, 544:112-119; incorporated herein by reference).

As used herein, the term “DNA” or “RNA” molecule or sequence (as well as sometimes the term “oligonucleotide”) refers to a molecule comprised generally of the deoxyribonucleotides or ribonucleotides, respectively, of adenine (A), guanine (G), thymine (T) and/or cytosine (C), wherein in “RNA”, T is replaced by uracil (U).

The terms “disorder”, “disease”, and “abnormal state” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. Certain characteristic signs, symptoms, and related factors can be quantitated through a variety of methods to yield important diagnostic information.

As used herein, a sample obtained at an “earlier time point” is a sample that was obtained at a sufficient time in the past such that relevant information could be obtained in the sample from the earlier time point as compared to the later time point. In certain embodiments, an earlier time point is at least two weeks earlier, at least four weeks earlier, at least six weeks earlier, at least two months earlier, at least three months earlier, at least six months earlier, at least nine months earlier, or at least one year earlier. Multiple subject samples (e.g., 3, 4, 5, 6, 7, or more) can be obtained at regular or irregular intervals over time and analyzed for trends in changes in marker levels. Appropriate intervals for testing for a particular subject can be determined by one of skill in the art based on ordinary considerations.

The term “expression” is used herein to mean the process by which an RNA or polypeptide is produced from DNA. The process involves the transcription of the gene into RNA if the marker of interest is an RNA, and further involves the translation of an mRNA into a polypeptide if the marker of interest a polypeptide or processing of the initial transcribed RNA into other subtypes of RNA, such as circRNA. Depending on the context in which used, “expression” may refer to the production of RNA, or protein, or both.

The term “expression level” is used herein to mean the level (e.g., amount) of an RNA or polypeptide present in a cell or biological sample.

A, “higher level”, “higher level of expression”, “higher level of induction” and the like of a marker refers to the marker's level in a sample that is greater than the standard error of the assay employed to assess the level, and is preferably at least 25% more, at least 50% more, at least 75% more, at least two, at least three, at least four, at least five, at least six, at least seven, at least eight, at least nine, or at least ten times the level of the marker in a control sample, and preferably the average level of the marker or markers in several control samples.

As used herein, a subject is “in need of” a treatment if such subject would benefit biologically, medically or in quality of life from such treatment (in some embodiments, a human).

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “level of expression of a gene”, “gene expression level”, “level of a marker”, and the like refer to the level of mRNA, as well as pre-mRNA nascent transcript(s), transcript processing intermediates, mature mRNA(s), non-encoding RNA products (e.g., circRNA) and degradation products, or the level of protein, encoded by the gene in the cell. The “level” of one of more biomarkers means the absolute or relative amount or concentration of the biomarkers in a sample.

A “lower level”, “lower level of expression” or “lower level of induction” of a marker refers to the marker's level in a sample that is less than 90%, 80%, 70%, 60%, 50%, 40%, 30%, 20%, or 10% of the level of the marker in a control sample, and preferably the average level of the marker in several control samples.

As used herein, “nucleic acid molecule” or “polynucleotides”, refers to a polymer of nucleotides. Non-limiting examples thereof include DNA (e.g., genomic DNA, cDNA), RNA molecules (e.g., mRNA, circRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions.

Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs). An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized.

As used herein, the term “obtaining” is understood herein as manufacturing, purchasing, or otherwise coming into possession of a physical entity or a value, e.g., a numerical value. “Obtaining a sample,” as the term is used herein, refers to obtaining possession of a sample, e.g., a tissue sample or cell sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third party laboratory that directly acquired the sample).

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural rare or synthetic nucleotides. They can be designed to enhance a chosen criteria like stability for example. Chimeras of deoxyribonucleotides and ribonucleotides may also be within the scope of the present invention.

As used herein, a “patient,” “subject” or “individual” are used interchangeably and refer to either a human or non-human animal. The term includes mammals such as humans. Typically, the animal is a mammal. A subject also refers to for example, non-human primates (e.g., monkey, male or female), cows, sheep, goats, horses, dogs, cats, rabbits, rats, mice, fish, birds and the like. In certain embodiments, the subject is a primate. In some embodiments, the subject is a human.

As used herein, the term “peripheral blood mononuclear cells,” abbreviated as “PBMCs”, refers to cells isolated from peripheral blood and identified as any blood cell with a round nucleus (i.e., lymphocytes, monocytes, natural killer cells (NK cells) or dendritic cells). PBMCs can be isolated from whole blood as follows, or through equivalent methods: The cell fraction corresponding to red blood cells and granulocytes (neutrophils, basophils and eosinophils) is removed from whole blood by density gradient centrifugation. A gradient medium with a density of ~1.077 g/ml separates whole blood into two fractions; PBMCs makes up the population of cells that remain in the low density fraction (upper fraction), whilst red blood cells and PMNs have a higher density and are found in the lower fraction. PBMCs include lymphocytes (T cells, B cells, and NK cells), monocytes, and dendritic cells. In humans, the frequencies of these populations vary across individuals, but typically, lymphocytes are in the range of 70-90%, monocytes from 10 to 20%, while dendritic cells are rare, accounting for only 1-2%. See Kleiveland et al. (Peripheral Blood Mononuclear Cells. In: Verhoeckx K, Cotter P, López-Expósito I, et al., editors. The Impact of Food Bioactives on Health: in vitro and ex vivo models [Internet]. Cham (CH): Springer; 2015. Chapter 15), incorporated herein by reference.

The phrase “pharmaceutically acceptable” indicates that the substance, composition or dosage form must be compatible chemically and/or toxicologically, with the other ingredients comprising a formulation, and/or the mammal being treated therewith.

As used herein, a “predetermined threshold value” or “threshold value” of a biomarker refers to the level of the biomarker (e.g., the expression level or quantity in a biological sample) in a corresponding control sample or group of control samples (e.g., an average level or mean level of the biomarker in the group of control samples). The predetermined threshold value may be determined prior to or concurrently with measurement of marker levels in a biological sample. The control sample may be from the same subject at a previous time or from different subjects.

As used herein, “prophylactic” or “therapeutic” treatment refers to administration to the subject of one or more agents or interventions to provide the desired clinical effect. If it is administered prior to clinical manifestation of the unwanted condition (e.g., disease or other unwanted state of the host animal) then the treatment is prophylactic, i.e., it protects the host against developing at least one sign or symptom of the unwanted condition, whereas if administered after manifestation of the unwanted condition, the treatment is therapeutic (i.e., it is intended to diminish, ameliorate, or maintain at least one sign or symptom of the existing unwanted condition or side effects therefrom).

As used herein, a “reference level” of a biomarker means a level of the biomarker that is indicative of therapeutic efficacy of a treatment, e.g., with a DHX9 inhibitor, or lack thereof. A “positive” reference level of a biomarker means a level that is indicative of a treatment, e.g., with a DHX9 inhibitor, having one or more therapeutic effects in a subject. A “negative” reference level of a biomarker means a level that is indicative of a lack of therapeutic effects from a treatment, e.g., with a DHX9 inhibitor.

As used herein, “sample” or “biological sample” includes a specimen or culture obtained from any source. Biological samples can be obtained from blood (including any blood product, such as whole blood, plasma, serum, or specific types of cells of the blood), interstitial fluid, lymph fluid, urine, saliva, and the like. Biological samples also include tissue samples, such as biopsy tissues or pathological tissues that have previously been fixed (e.g., formaline snap frozen, cytological processing, etc.). In an embodiment, the biological sample is from blood. In another embodiment, the biological sample is a biopsy tissue from a tumor.

The term “therapeutic effect” refers to a local or systemic effect in animals, particularly mammals, and more particularly humans caused by a pharmacologically active substance. The term thus means any substance intended for use in the diagnosis, cure, mitigation, treatment, or prevention of disease, or in the enhancement of desirable physical or mental development and conditions in an animal or human. A therapeutic effect can be understood as a decrease in tumor growth, decrease in tumor growth rate, stabilization or decrease in tumor burden, stabilization or reduction in tumor size, stabilization or decrease in tumor malignancy, increase in tumor apoptosis, and/or a decrease in tumor angiogenesis.

As used herein, “therapeutically effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment, e.g., is sufficient to ameliorate at least one sign or symptom of the disease, e.g., to prevent progression of the disease or condition, e.g., prevent tumor growth, decrease tumor size, induce tumor cell apoptosis, reduce tumor angiogenesis, prevent metastasis. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. For example, certain compounds discovered by the methods of the present invention may be administered in a sufficient amount to produce a reasonable benefit/risk ratio applicable to such treatment. Administration of a therapeutically effective amount of a compound may require the administration of more than one dose of the compound.

As used herein, the term “treat”, “treating” or “treatment” of any disease, condition or disorder, refers to the management and care of a patient for the purpose of combating the disease, condition, or disorder and includes the administration of a compound of the present disclosure to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### II. Methods and Assays

CircRNAs are present in small amounts in biological samples, and thought to be not abundant enough to allow for their use as meaningful biomarkers. The methods and assays described herein are based, in part, on the surprising findings that levels of Alu-mediated circular RNAs are detectable in biological samples and that their changes correlate with DHX9 inhibition in vitro and in vivo. Thus, provided herein are assays and methods for detecting, measuring, and monitoring activity of a DHX9 inhibitor in vitro and/or in vivo.

In some aspects, the present disclosure provides methods for detecting inhibition of DHX9 in a cell, or a population of cells, comprising contacting the cell or population of cells with a DHX9 inhibitor, and detecting or measuring the level of an Alu-mediated circular RNA (Alu-circRNA) in the cell or population of cells.

In some embodiments, the cells are maintained in cell culture in vitro. In some embodiments, the cell is from a primary cell line. In some embodiments, the cell is from an established or immortalized cell line.

In some embodiments, the cell is an ex vivo cell isolated from a subject. In some embodiments, the cell is isolated from a biological sample from a subject, e.g., blood, interstitial fluid, lymph fluid, organ tissue, biopsy tissue, and skin tissue. In some embodiments, the cell is contacted with the DHX9 in vivo.

In some embodiments, the cell is a normal, non-cancerous cell. In some embodiments, the cell is a blood cell. In preferred embodiments, the cell is a peripheral blood mononuclear cell (PBMC). In preferred embodiments, the population of cells comprises or consists of PBMCs.

In some embodiments, the cell or population of cells are a cancer cell. In some embodiments, the cancer is selected from the group consisting of, but not limited to, colorectal, endometrial, ovarian, gastric, hematopoietic, breast, brain, skin, lung, blood, prostate, head and neck, pancreatic, bladder, bone, soft tissue, kidney, liver, and Ewing's sarcoma.

In some embodiments, the cancer cell is derived from a cancer that has microsatellite instability (MSI). In some embodiments, the cancer cell is characterized to have genomic instability. In some embodiments, the cancer cell is defective in mismatch repair. In some embodiments, cancer cell having genomic instability is selected for use in the methods and assays of the disclosure.

In some embodiments, the cancer cell is isolated from a tumor from a subject. In some embodiments, the cancer cell is isolated from the blood of the a subject having a hematopoeitic cancer.

In some embodiments, the cells are contacted with one or more amounts or concentrations of a DHX9 inhibitor, and the levels of the Alu-circRNA is measured or determined for each amount or concentration of the inhibitor.

In some embodiments, the cells are contacted with a DHX9 inhibitor for a period of time sufficient for the inhibitor to inhibit DHX9 expression. In some embodiments, the cells are contacted with a DHX9 inhibitor for a period of time sufficient for the inhibitor to inhibit DHX9 activity. In some embodiments, the cell or populations of cells is contacted with a DHX9 inhibitor for a period of time sufficient for the Alu-circRNA to be produced, e.g., to detectable levels.

In some embodiments, the level of the Alu-circRNA is measured or determined between 1 to 24 hours, between 2 to 24 hours, between 4 to 24 hours, between 6 to 24 hours, between 8 to 24 hours, between 10 to 24 hours, between 12 to 24 hours, between 14 to 24 hours, between 16 to 24 hours, between 18 to 24 hours, between 20 to 24 hours, between 22 to 24 hours, between 1 to 20 hours, between 2 to 20 hours, between 4 to 20 hours, between 6 to 20 hours, between 8 to 20 hours, between 10 to 20 hours, between 12 to 20 hours, between 14 to 20 hours, between 16 to 20 hours, between 18 to 20 hours, between 1 to 16 hours, between 2 to 16 hours, between 4 to 16 hours, between 6 to 16 hours, between 8 to 16 hours, between 10 to 16 hours, between 12 to 16 hours, between 14 to 16 hours, between 1 to 12 hours, between 2 to 12 hours, between 4 to 12 hours, between 6 to 12 hours, between 8 to 12 hours, between 10 to 12, 1 to 8 hours, between 2 to 8 hours, between 4 to 8 hours, or between 6 to 8 hours after the cell is contacted with the DHX9 inhibitor.

In some embodiments, the level of the Alu-circRNA is measured or determined at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42 hours, 48 hours, 54 hours, 60 hours, 66 hours, 72 hours, 78 hours, 84 hours, 90 hours, 96 hours, 102 hours, 108 hours, 114 hours or 120 hours after the cell is contacted with the DHX9 inhibitor.

In some embodiments, the level of the Alu-circRNA is measured or determined at least 1 day, at least 1.5 days, at least 2 days, at least 2.5 days, at least 3 days, at least 3.5 days, at least 4 days, at least 4.5 days, at least 5 days, at least 5.5 days, at least 6 days, at least 6.5 days, or at least 7 days after the cell is contacted with the DHX9 inhibitor. In some embodiments, the level of the Alu-circRNA is measured or determined about 1 day, about 1.5 days, about 2 days, about 2.5 days, about 3 days, about 3.5 days, about 4 days, about 4.5 days, about 5 days, about 5.5 days, about 6 days, about 6.5 days, or about 7 days after the cell is contacted with the DHX9 inhibitor.

In some embodiments, the cells are contacted with a DHX9 inhibitor, and the levels of the Alu-circRNA is measured or determined at multiple different times after the cells are first contacted with the inhibitor. In some embodiments, the methods further comprise measuring or determining the level of the Alu-circRNA in cells that have not been contacted, or prior to contact, with the DHX9 inhibitor, and this level of the Alu-circRNA is used to establish a control level.

In other aspects, the present disclosure generally provides methods for detecting or measuring inhibition of DHX9 in a subject. The present disclosure further generally provides methods for detecting engagement of DHX9 by a DHX9 inhibitor in a subject. The methods comprise detecting or measuring the level of an Alu-circRNA in a biological sample obtained from subject. The subject is scheduled to be administered, is undergoing treatment with, or has recently been administered a DHX9 inhibitor. In some embodiments, the sample is obtained from the subject following administration of a DHX9 inhibitor. In some embodiments, the method further comprises administering a DHX9 inhibitor to the subject.

The present disclosure provides a method for measuring or detecting activity of a DHX9 inhibitor in a subject, comprising administering a DHX9 inhibitor to the subject, and measuring or detecting the level of an Alu-circRNA in a biological sample from the subject.

The present disclosure also provides a method for measuring or detecting engagement of DHX9 by a DHX9 inhibitor in a subject, comprising administering a DHX9 inhibitor to the subject, and measuring or detecting the level of an Alu-circRNA in a biological sample from the subject.

The present disclosure also provides a method for measuring or detecting activity of a DHX9 inhibitor in a subject, comprising measuring or detecting the level of an Alu-circRNA in a biological sample from the subject, wherein the subject is being treated with (i.e., has been administered) the DHX9 inhibitor. In some embodiments, the method further comprises the step of administering the DHX9 inhibitor to a subject. The present disclosure also provides a method for measuring or detecting engagement of DHX9 by a DHX9 inhibitor in a subject, comprising measuring or detecting the level of an Alu-circRNA in a biological sample from the subject, wherein the subject is being treated with (i.e., has been administered) the DHX9 inhibitor. In some embodiments, the method further comprises the step of administering the DHX9 inhibitor to a subject.

The present disclosure also provides a method for monitoring the treatment of a subject with a DHX9 inhibitor, comprising obtaining information as to the level of Alu-circRNA in a biological sample obtained from the subject after administration of the DHX9 inhibitor. In some embodiments, the method further comprises the step of administering the DHX9 inhibitor to the subject.

In certain embodiments, the subject is a mammal, such as a human, a monkey, a cow, a sheep, a goat, a horse, a dog, a cat, a rabbit, a rat, or a mice. In some embodiments, the subject is a human.

In some embodiments, the subject is a healthy, normal subject. In some embodiments, the subject has a cancer. In some embodiments, the subject is an animal model of a cancer, e.g., a mouse having a human xenograft tumor.

In some embodiments, the cancer is a microsatellite instability (MSI) cancer. In some embodiments, the cancer is microsatellite-instability high (MSI-H). In some embodiments, the cancer is defective in mismatch repair.

In some embodiments, the cancer is selected from the group consisting of colorectal cancer, endometrial cancer, ovarian cancer, gastric cancer, hematopoietic cancer, breast cancer, brain cancer, skin cancer, lung cancer, blood cancer, prostate cancer, head and neck cancer, pancreatic cancer, bladder cancer, bone cancer, soft tissue cancer, kidney cancer, liver cancer, and Ewing's sarcoma. In some embodiments, the cancer is colorectal cancer. In some embodiments, the cancer is colorectal cancer with microsatellite instability (MSI-CRC).

In certain embodiments, the biological sample is selected from the group consisting of blood, interstitial fluid, lymph fluid, organ tissue, biopsy tissue, and skin tissue. In some embodiments, the biological sample is blood. In some embodiments, the biological sample is cells isolated from blood.

In some embodiments, the biological sample is peripheral blood mononuclear cells (PBMC). In some embodiments, the biological sample comprises peripheral blood mononuclear cells (PBMC). In some embodiments, the biological sample consists of peripheral blood mononuclear cells (PBMC).

In some embodiments, the methods further comprise obtaining the biological sample from the subject. In some embodiments, the methods further comprise isolating a certain cell population from the biological sample. In some embodiments, the methods may further comprise isolating PBMCs from a blood sample. The cell population can be isolated from the biological sample based on physical properties (e.g., size, density, buoyancy), or based on specific biomarkers.

In some embodiments, the methods comprise measuring or determining the level of the Alu-circRNA in a whole blood sample. In some embodiments, the methods comprise measuring or determining the level of the Alu-circRNA in a certain cell population of the biological sample, e.g., PBMCs in a blood sample. In some embodiments, the methods comprise measuring the level of the Alu-circRNA in PBMCs.

In some embodiments, the DHX9 inhibitor is administered more than once to the subject, i.e., multiple doses of the DHX9 inhibitor are administered to the subject.

In some embodiments, the biological sample is obtained from the subject following administration of the DHX9 inhibitor. In some embodiments, the biological sample is obtained after administration of multiple doses of the DHX9 inhibitor. In some embodiments, the level of the Alu-cirRNA is measured in biological samples obtained from the subject at more than one time point. In some embodiments, the biological sample is obtained following each administration of the DHX9 inhibitor. In some embodiments, biological samples are obtained from the subject at multiple time points after administration of a dose of the DHX9 inhibitor.

In some embodiments, the biological sample is obtained between 1 to 24 hours, between 2 to 24 hours, between 4 to 24 hours, between 6 to 24 hours, between 8 to 24 hours, between 10 to 24 hours, between 12 to 24 hours, between 14 to 24 hours, between 16 to 24 hours, between 18 to 24 hours, between 20 to 24 hours, between 22 to 24 hours, between 1 to 20 hours, between 2 to 20 hours, between 4 to 20 hours, between 6 to 20 hours, between 8 to 20 hours, between 10 to 20 hours, between 12 to 20 hours, between 14 to 20 hours, between 16 to 20 hours, between 18 to 20 hours, between 1 to 16 hours, between 2 to 16 hours, between 4 to 16 hours, between 6 to 16 hours, between 8 to 16 hours, between 10 to 16 hours, between 12 to 16 hours, between 14 to 16 hours, between 1 to 12 hours, between 2 to 12 hours, between 4 to 12 hours, between 6 to 12 hours, between 8 to 12 hours, between 10 to 12, 1 to 8 hours, between 2 to 8 hours, between 4 to 8 hours, or between 6 to 8 hours after administration of the DHX9 inhibitor. In some embodiments, the biological sample is obtained at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, 42 hours, 48 hours, 54 hours, 60 hours, 66 hours, 72 hours, 78 hours, 84 hours, 90 hours, 96 hours, 102 hours, 108 hours, 114 hours or 120 hours after administration of the DHX9 inhibitor. In some embodiments, the level of the Alu-circRNA is measured or determined at least 0.5 days, at least 1 day, at least 1.5 days, at least 2 days, at least 2.5 days, at least 3 days, at least 3.5 days, at least 4 days, at least 4.5 days, at least 5 days, at least 5.5 days, at least 6 days, at least 6.5 days, or at least 7 days after administration of the DHX9 inhibitor.

In some embodiments, the biological sample is obtained about 0.5 days, about 1 day, about 1.5 days, about 2 days, about 2.5 days, about 3 days, about 3.5 days, about 4 days, about 4.5 days, about 5 days, about 5.5 days, about 6 days, about 6.5 days, or about 7 days after administration of the DHX9 inhibitor.

In some embodiments, the methods further comprise obtaining a biological sample from the subject prior to administration of the DHX9 inhibitor, and the level of the Alu-circRNA in this sample is used to establish a control level.

In certain embodiments, the DHX9 inhibitor is administered to the subject orally, subcutaneously, or intravenously.

It will be appreciated that the measurements of the level of the Alu-circRNA obtained from the methods and assays of the disclosure are useful for evaluating the activity or efficacy of a DHX9 inhibitor in vitro and in vivo. For example, in some embodiments, the level of the Alu-circRNA determined using the methods of the present disclosure is correlated (e.g., directly correlated) to inhibition of DHX9 activity by the DHX9 inhibitor in the cell or in the subject. In some embodiments, the level of the Alu-circRNA is correlated (e.g., directly correlated) to inhibition of the ATPase activity of DHX9. In some embodiments, the level of the Alu-circRNA is correlated (e.g., directly correlated) to inhibition of the resolvase activity of DHX9. In some embodiments, the level of the Alu-circRNA is correlated to the inhibition of proliferation of a cancer cell, for example an MSI cancer cell, by the DHX9 inhibitor. In some embodiments, the level of the Alu-circRNA is correlated to the inhibition of growth of an MSI cancer in a subject by the DHX9 inhibitor. In some embodiments, the level of the Alu-circRNA is not correlated to the inhibition of growth of PBMCs in a subject by the DHX9 inhibitor. In various embodiments, the cell can be an isolated cell, a cell in vitro, a cell ex vivo, or a cell in vivo in a subject.

It will further be appreciated that the level of Alu-circRNA determined by the methods and assays of the instant disclosure is useful to determine if a DHX9 inhibitor (e.g., a candidate inhibitor) is engaging the DHX9 target, and/or if the DHX9 inhibitor is effective at inhibiting DHX9 activity, in a cell or in a subject.

In a non-limiting example, the Alu-circRNA level determined for a cell or cell population after treatment with a candidate DHX9 inhibitor is compared to the Alu-circRNA level determined for a control cell or cell population not treated with a DHX9 inhibitor (e.g., a control level). If the level of the Alu-circRNA in the treated cells is increased as compared to the level of the Alu-circRNA in the control cells, the candidate DHX9 inhibitor is determined to engage the DHX9 target, and/or to inhibit DHX9 activity, in the cells.

Similarly, the Alu-circRNA level determined for a biological sample obtained from a subject after treatment with a candidate DHX9 inhibitor is compared to the Alu-circRNA level determined for a control sample from a subject not treated with a DHX9 inhibitor (e.g., a control level). If the level of the Alu-circRNA in the sample obtained after treatment with the candidate DHX9 inhibitor is increased as compared to the level of the Alu-circRNA in the control sample, the candidate DHX9 inhibitor is determined to engage the DHX9 target, and/or to inhibit DHX9 activity, in the subject.

It will be further appreciated that Alu-circRNA levels can be determined and compared among different candidate DHX9 inhibitors to identify a candidate inhibitor that is more or less effective at engaging the DHX9 target and/or inhibiting DHX9 activity in vitro or in vivo. In some embodiments, if the same dosing regimen is utilized for two or more inhibitors, an inhibitor that induces a higher Alu-circRNA level may be considered to be more effective at engaging the DHX9 target and/or inhibiting DHX9 activity than an inhibitor that induces a lower Alu-circRNA level, and an inhibitor that induces a lower Alu-circRNA level may be considered to be less effective at engaging the DHX9 target and/or inhibiting DHX9 activity than an inhibitor that induces a higher Alu-circRNA level.

In some embodiments, reference levels can be established for an Alu-cirRNA marker, and levels of the Alu-circRNA induced by a DHX9 inhibitor can be compared to the reference levels to make a determination of DHX9 engagement and/or efficacy of the DHX9 inhibitor.

In certain embodiments, the level of Alu-circRNA determined using the methods and assays of the instant disclosure is useful in determining an appropriate dose, e.g., a therapeutically effective dose or a minimally effective dose, of the DHX9 inhibitor. The level of Alu-circRNA determined using the methods and assays of the instant disclosure is also useful in determining an effective therapeutic treatment regimen for the DHX9 inhibitor.

### DHX9 Inhibitors

The methods provided in the present disclosure are applicable to any DHX9 inhibitors, and are not limited to the exemplary DHX9 inhibitors described herein. In some embodiments the DHX9 inhibitors are previously known in the art, and have been shown to reduce or inhibit the expression or activity of DHX9 in vitro or in vivo. In some embodiments the DHX9 inhibitors are candidate DHX9 inhibitors under discovery and testing for their ability to reduce or inhibit expression or activity of DHX9 in vitro or in vivo.

In some embodiments, the DHX9 inhibitor is selected from a small molecule, an antisense oligonucleotide, or an RNA interfering agent such as small interfering RNA (siRNA), a small hairpin RNA (shRNA), a microRNA (miRNA), or a piwiRNA (piRNA).

In some embodiments, the DHX9 inhibitor is a small molecule. A small molecule inhibitor is typically an organic compound with low molecular weight that has high cell penetration.

In some embodiments, the small molecule inhibitor of DHX9 is any DHX9 small molecule inhibitor found in the art. Exemplary DHX9 small molecule inhibitors and methods of synthesis thereof can be found at least in international PCT applications PCT/US2023/013303 and PCT/US2023/012929, the entire contents of each of which are incorporated herein by reference.

In some embodiments, the DHX9 inhibitor is selected from the compounds listed in Table 1.

TABLE 1
Exemplary DHX9 small molecule inhibitors

| Compound | Structure |
|---|---|
| Compound 1: N-(3-chloro-5- methanesulfon- amidophenyl)- 4-(3-methyl- pyridin-2-yl)- thiophene-2- carboxamide | 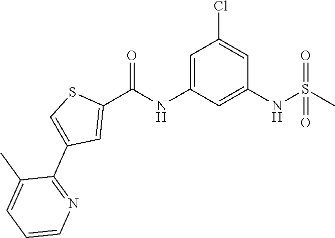 |
| Compound 2: N-(3-chloro-5- methanesulfon- amidophenyl)- 5-(3,5-difluoro- pyridin-2-yl)- 1-methyl-1H- pyrrole-3- carboxamide | 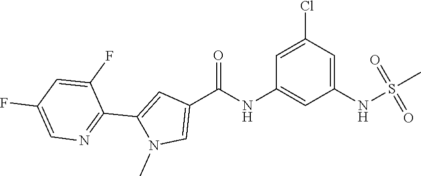 [SMILES: Cn1cc(C(=O)Nc2cc(Cl)cc(NS(C)(=O)=O)c2)cc1-c1ncc(F)cc1F] |
| Compound 3 N-(3-chloro-5- (methylsulfon- amido)phenyl)- 4-(5-isoprop- oxypyrimidin- 2-yl)-5-methyl- thiophene-2- carboxamide | 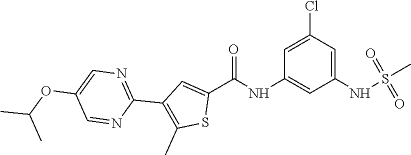 [SMILES: Cc1sc(C(=O)Nc2cc(Cl)cc(NS(C)(=O)=O)c2)cc1-c1ncc(OC(C)C)cn1] |
| Compound 4 4-(5-(tert- butoxy)pyrim- idin-2-yl)-N- (3-chloro-5- (methylsulfon- amido)phenyl)- 5-methylthio- phene-2-carbox- amide | 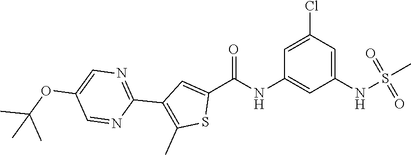 [SMILES: Cc1sc(C(=O)Nc2cc(Cl)cc(NS(C)(=O)=O)c2)cc1-c1ncc(OC(C)(C)C)cn1] |
| Compound 7 4-bromo-N-(3- (methylsulfon- amido)phenyl)- thiophene-2- carboxamide | 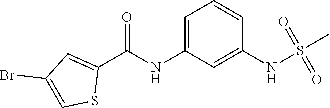 [SMILES: CS(=O)(=O)Nc1cccc(NC(=O)c2cc(Br)cs2)c1] |

Methods for preparing the compounds listed in Table 1 are provided below.
Abbreviations:
DMSO = dimethylsulfoxide
HATU = 1-[Bis(dimethylamino)methylene]-1H-1,2,3-triazolo[4,5-b]pyridinium 3-oxide hexafluorophosphate
HPLC = high pressure liquid chromatography
LCMS = liquid chromatography mass spectrometry
TFA = Trifluoroacetic acid
AcOK = KOAc = potassium acetate
Pd(dppf)Cl₂ = [1,1′-Bis(diphenylphosphino)ferrocene]dichloropalladium(II)
K₂CO₃ = potassium carbonate
LiOH = lithium hydroxide
TCFH = N′-tetramethylformamidinium hexafluorophosphate
K₃PO₄ = potassium phosphate
tBuOH = tert-butanol
HCl = hydrochloric acid
NMI = N-methylimidazole
ACN = acetonitrile
DCM = dichloromethane
DMF = N,N-dimethylformamide
EA = ethyl acetate
PE = petroleum ether
H₂O = water
EtOH = ethanol

### General Methods

1. ¹H NMR spectra were recorded on any of the following:
   NMR10 Bruker AVANCE III HD 300 MHz
   NMR16 Bruker AVANCE III HD 300 MHz
   NMR19 Bruker AVANCE III HD 400 MHz
   NMR24 Bruker AVANCE NEO 400 MHz
   NMR30 Bruker AVANCE NEO 400 MHz
   Bruker AVβ 400
2. LCMS measurement was run on SHIMADZU LCMS-2020 or Agilent 1200 HPLC/6100 SQ System using the follow conditions:
   Method A: Mobile Phase: A: Water (0.05% TFA) B: Acetonitrile (0.05% TFA); Gradient Phase: 5% B to 100% B within 2.0 min, 100% B with 0.7 min (total runtime: 2.8 min); Flow Rate: 1.5 mL/min; Column: HALO C18, 3.0*30 mm, 2.0 μm; Column Temperature: 40° C. Detectors: AD2 ELSD, PDA (220 nm and 254 nm), ESI.
   Method B: Mobile Phase: A: Water (0.1% FA) B: Acetonitrile (0.1% FA); Gradient Phase: 5% B to 100% B within 2.0 min, 100% B with 0.7 min (total runtime: 2.8 min); Flow Rate: 1.5 mL/min; Column: HALO C18, 3.0*30 mm, 2.0 μm; Column Temperature: 40° C. Detectors: AD2 ELSD, PDA (220 nm and 254 nm), ESI.
   Method C: Mobile Phase: A: Water (5 mM NH₄HCO₃) B: Acetonitrile; Gradient Phase: 10% B to 95% B within 2.0 min, 100% B with 0.6 min (total runtime: 2.8 min); Flow Rate: 1.5 mL/min; Column: Poroshell HPH-C18, 3.0*50 mm, 4.0 μm; Column Temperature: 40° C. Detectors: AD2 ELSD, PDA (220 nm and 254 nm), ESI.
   Method D: Mobile Phase: A: Water (0.01% TFA) B: Acetonitrile (0.01% TFA); Gradient Phase: 5% B to 95% B within 1.4 min, 95% B with 1.6 min (total runtime: 3 min); Flow Rate: 2.0 mL/min; Column: SunFire C18, 4.6*50 mm, 3.5 μm; Column Temperature: 40° C. Detectors: ADC ELSD, DAD (214 nm and 254 nm), ES-API.

### Preparation of Intermediate A

The compounds claimed herein were prepared following the procedures outlined in the following schemes. Compound names were generated using the software built into ChemDraw. To the extent that there are discrepancies between the name of a compound and its depicted structure, the depicted chemical structure is to be taken as the appropriate compound.

### Intermediate A: N-(3-amino-5-chlorophenyl)methanesulfonamide

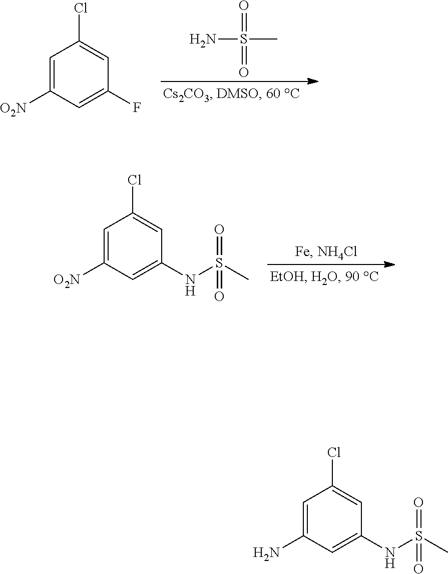

Step 1: To a stirred mixture of 1-chloro-3-fluoro-5-nitrobenzene (45 g, 256.35 mmol, 1.00 equiv) and methane sulfonamide (24.38 g, 256.35 mmol, 1.00 equiv), Cs₂CO₃ (250.57 g, 769.05 mmol, 3.00 equiv) in 500 mL of DMSO, the resulted solution was stirred for 2 h at 60° C. The mixture was cooled then quenched with 1000 mL of water and extracted with 3×1000 mL of ethyl acetate and dried over anhydrous Na₂SO₄ and concentrated and the residue was purified onto silica gel column eluted with 50% of ethyl acetate in petroleum ether to afford N-(3-chloro-5-nitrophenyl) methane sulfonamide (41 g, 63.81%) as a light yellow solid. LCMS (ESI) [M+H]⁺: 249.9

Step 2: To a stirred solution of N-(3-chloro-5-nitrophenyl) methanesulfonamide (41.00 g, 163.57 mmol, 1.00 equiv) and Fe (91.35 g, 1635.75 mmol, 10.00 equiv), NH₄Cl (87.50 g, 1635.75 mmol, 10.00 equiv) in 500 mL of ethanol and 300 mL of water, this was stirred for 2 h at 90° C. The resulting mixture was filtered, the filter cake was washed with 6×50 mL of methanol. The filtrate was concentrated under reduced pressure. The residue was purified onto silica gel column eluted with 50% of ethyl acetate in petroleum ether to afford N-(3-amino-5-chlorophenyl) methanesulfonamide (25.3 g, 70.1%) as a light yellow solid. LCMS (ESI) [M+H]⁺: 220.01

¹H NMR (300 MHz, DMSO-d₆) δ 9.64 (s, 1H), 6.41-6.31 (m, 3H), 5.54 (s, 2H), 2.98 (s, 3H)

### Intermediate E: N-(3-amino-5-chlorophenyl)propane-]-sulfonamide

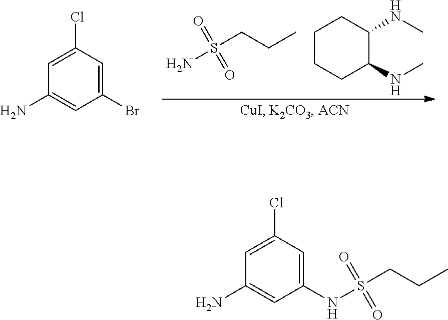

A solution of 3-bromo-5-chloroaniline (1 g, 4.843 mmol, 1 equiv), propane-1-sulfonamide (1.19 g, 9.686 mmol, 2 equiv), (1S,2S)—N¹,N²-dimethylcyclohexane-1,2-diamine (2.07 g, 14.529 mmol, 3 equiv), CuI (2.77 g, 14.529 mmol, 3 equiv) and K₂CO₃ (2.01 g, 14.529 mmol, 3 equiv) in ACN (10 mL) was stirred for 2 hours at 110° C. under nitrogen atmosphere. The resulting mixture was concentrated under reduced pressure. The residue was purified by silica gel column chromatography, eluted with PE/EA (1:1) to afford N-(3-amino-5-chlorophenyl)propane-1-sulfonamide (540 mg, 45% yield) as a yellow solid. LCMS (ESI) [M+H]⁺: 249

### Compound 1: N-(3-chloro-5-methanesulfonamidophenyl)-4-(3-methylpyridin-2-yl)thiophene-2-carboxamide

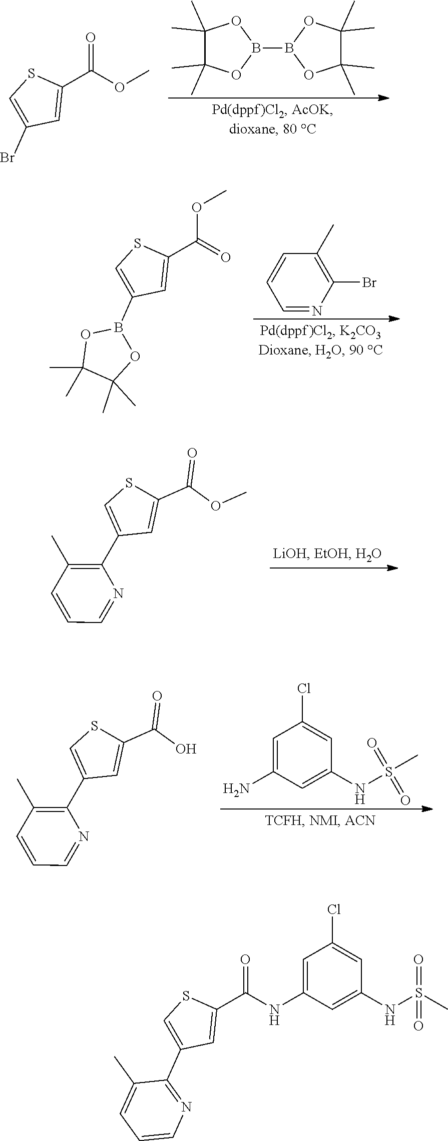

Step 1: A solution of methyl 4-bromothiophene-2-carboxylate (150 g, 678.52 mmol, 1.00 equiv), Pd(dppf)Cl₂ (24.82 g, 33.93 mmol, 0.05 equiv), AcOK (199.77 g, 2035.55 mmol, 3.00 equiv) and bis(pinacolato)diboron (206.76 g, 814.22 mmol, 1.2 equiv) in 1000 mL of dioxane. This was stirred for 2 hours at 80° C. under nitrogen atmosphere. The resulting mixture was cooled and concentrated. The residue was purified onto silica gel column eluted with 10% of ethyl acetate in petroleum ether to afford methyl 4-(4,4,5,5-tetramethyl-1,3,2-dioxaborolan-2-yl) thiophene-2-carboxylate (170 g, 92.5%) as a yellow solid.

Step 2: A solution of methyl 4-(4,4,5,5-tetramethyl-1,3,2-dioxaborolan-2-yl)thiophene-2-carboxylate (170 g, 634.02 mmol, 1.00 equiv), Pd(dppf)Cl₂ (23.20 g, 31.70 mmol, 0.05 equiv), K₂CO₃ (262.87 g, 1902.06 mmol, 3.00 equiv) and 2-bromo-3-methylpyridine (109.07 g, 634.02 mmol, 1.00 equiv) in 1000 mL of 1,4-dioxane and 200 mL of water, this was stirred for 2 hours at 90° C. under nitrogen atmosphere. The resulting mixture was cooled and concentrated. The residue was purified onto silica gel column chromatography, eluted with 50% of ethyl acetate in petroleum ether to afford methyl 4-(3-methylpyridin-2-yl)thiophene-2-carboxylate (115 g, 77%) as a yellow solid. LCMS (ESI) [M+H]+: 234

Step 3: To a stirred solution of methyl 4-(3-methylpyridin-2-yl)thiophene-2-carboxylate (115 g, 492.95 mmol, 1.00 equiv), LiOH (118.06 g, 4929.49 mmol, 10.00 equiv) in 250 mL of ethanol and 250 mL of water. Then the mixture was stirred for 2 hours at room temperature. The mixture was adjusted pH to 5 with citric acid. The precipitated solids were collected by filtration and washed with water. The resulted solid was dried under vacuum to afford 4-(3-methylpyridin-2-yl)thiophene-2-carboxylic acid (60 g, 54.9%) as a yellow solid.

LCMS (ESI) [M+H]+: 220

Step 4: To a stirred solution of 4-(3-methylpyridin-2-yl)thiophene-2-carboxylic acid (60 g, 273.65 mmol, 1.00 equiv), TCFH (115.17 g, 410.47 mmol, 1.50 equiv) and NMI (67.40 g, 820.94 mmol, 3.00 equiv) in 1000 mL of acetonitrile, to this was added N-(3-amino-5-chlorophenyl)methanesulfonamide (50 g, 226.58 mmol, 0.83 equiv).Then the mixture was stirred for 2 hours at room temperature. The mixture was concentrated and purified by reverse flash chromatography eluting with 56% acetonitrile in water (0.05% FA) to afford N-(3-chloro-5-methanesulfonamidophenyl)-4-(3-methylpyridin-2-yl)thiophene-2-carboxamide (49.6634 g, 42.8%) as a white solid. LCMS (ESI) [M+H]+: 422. ¹H NMR (300 MHz, Methanol-d4) δ 8.49-8.41 (m, 1H), 8.21 (d, J=1.4 Hz, 1H), 7.99 (d, J=1.4 Hz, 1H), 7.85-7.75 (m, 1H), 7.69 (t, J=1.9 Hz, 1H), 7.60 (t, J=1.9 Hz, 1H), 7.35 (dd, J=7.7, 4.9 Hz, 1H), 7.05 (t, J=2.0 Hz, 1H), 3.06 (s, 3H), 2.50 (s, 3H).

### Compound 2: N-(3-chloro-5-methanesulfonamidophenyl)-5-(3,5-difluoropyridin-2-yl)-1-methyl-1H-pyrrole-3-carboxamide

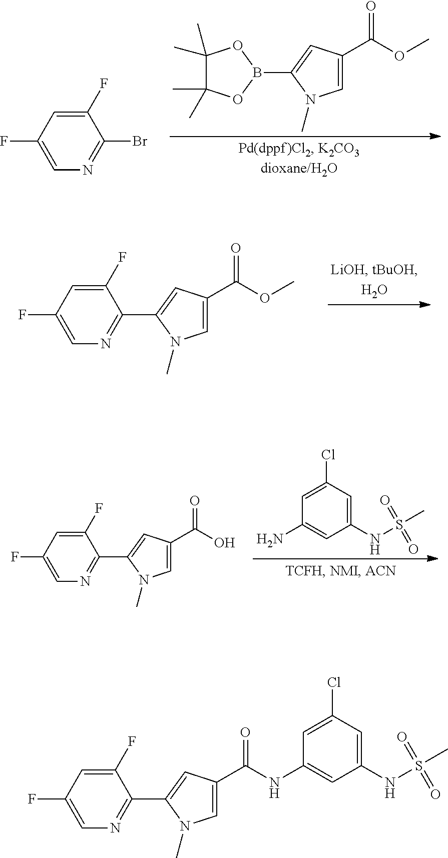

Step 1: To a mixture of methyl 1-methyl-5-(4,4,5,5-tetramethyl-1,3,2-dioxaborolan-2-yl)-1H-pyrrole-3-carboxylate (116 g, 440 mmol, 1 equiv), 2-bromo-3,5-difluoropyridine (85 g, 440 mmol, 1 equiv), Pd(dppf)Cl₂ (18 g, 22 mmol, 0.05 equiv) and K₃PO₄ (200 g, 880 mmol, 2 equiv) was added dioxane (1 L) and H₂O (100 mL). The resulting mixture was stirred for 2 hours at 80° C. under N₂ atmosphere. The reaction mixture was extracted with ethyl acetate. The organic phase was concentrated under reduced pressure. The residue was purified by flash chromatography on silica gel eluting with 30% of ethyl acetate in petroleum ether to afford methyl 5-(3,5-difluoropyridin-2-yl)-1-methyl-1H-pyrrole-3-carboxylate (98 g, 88.6% yield) as a white solid.

Step 2: To a stirred solution of methyl 5-(3,5-difluoropyridin-2-yl)-1-methyl-1H-pyrrole-3-carboxylate (295 g, 1165 mmol, 1 equiv) in tBuOH (3 L) and water (2 L) was added LiOH (246 g, 5853 mmol, 5 equiv, in 1 L water). The mixture was stirred for 4 hours at 60° C. The pH value of the solution was adjusted to 3 with 1M of HCl (aq). The product was precipitated from the solution. The mixture was filtered and the filter cake was washed with water (3×200 mL) to afford 5-(3,5-difluoropyridin-2-yl)-1-methyl-1H-pyrrole-3-carboxylic acid (212 g, 76.0% yield) as a white solid. LCMS [M+H]⁺: 239.

Step 3: A mixture of 5-(3,5-difluoropyridin-2-yl)-1-methyl-1H-pyrrole-3-carboxylic acid (212 g, 886 mmol, 1 equiv), TCFH (341 g, 1219 mmol, 1.37 equiv), NMI (199 g, 2438 mmol, 2.75 equiv) and N-(3-amino-5-chlorophenyl)methanesulfonamide (214 g, 487 mmol) in ACN (2 L) was stirred for two hours at room temperature. The mixture was quenched with water (2 L). The resulting mixture was extracted with DCM (3×3 L) and water. The combined organic layers were dried over anhydrous Na₂SO₄. After filtration, the filtrate was concentrated under reduced pressure. The residue was purified by silica gel column chromatography, eluted with DCM/ACN (5:1) to afford N-(3-chloro-5-methanesulfonamidophenyl)-5-(3,5-difluoropyridin-2-yl)-1-methyl-1H-pyrrole-3-carboxamide (155 g, 39.5% yield) as a white solid. LCMS [M+H]⁺: 441. ¹H NMR (400 MHz, DMSO-d₆) δ 10.00 (s, 1H), 9.90 (s, 1H), 8.59 (d, J=2.4 Hz, 1H), 8.08 (ddd, J=11.1, 8.9, 2.5 Hz, 1H), 7.79-7.69 (m, 2H), 7.64 (t, J=1.9 Hz, 1H), 7.21 (dd, J=3.6, 1.9 Hz, 1H), 6.91 (t, J=2.0 Hz, 1H), 3.89 (s, 3H), 3.06 (s, 3H).

### Compound 3: N-(3-chloro-5-(methylsulfonamido)phenyl)-4-(5-isopropoxypyrimidin-2-yl)-5-methylthiophene-2-carboxamide

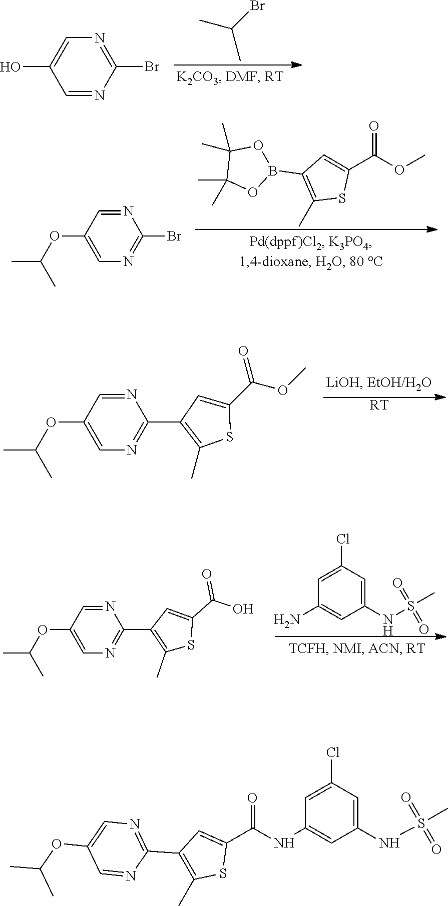

Step 1: A mixture of 2-bromopyrimidin-5-ol (15.0 g, 85.7 mmol, 1 equiv), K₂CO₃ (35.48 g, 257.1 mmol, 3 equiv) and 2-bromopropane (13.71 g, 111.5 mmol, 1.3 equiv) in DMF (150 mL) was stirred for 5 hours at room temperature. The reaction mixture was diluted with water (1 L). The resulting solution was extracted with EA (3×800 mL) and the organic layers were combined. The organic layer was dried over anhydrous sodium sulfate and concentrated under vacuum. The residue was purified by flash chromatography on silica gel column eluting with EA/PE (20%) to afford 2-bromo-5-isopropoxypyrimidine (15.0 g, 81% yield) as a white solid. LCMS (ESI) [M+H]⁺: 217

Step 2: A mixture of 2-bromo-5-isopropoxypyrimidine (14.0 g, 64.5 mmol, 1 equiv), Pd(dppf)Cl₂ (3.3 g, 4.5 mmol, 0.07 equiv), K₃PO₄ (41.1 g, 193.5 mmol, 3 equiv) and methyl 5-methyl-4-(4,4,5,5-tetramethyl-1,3,2-dioxaborolan-2-yl) thiophene-2-carboxylate (23.66 g, 83.8 mmol, 1.3 equiv) in 1,4-dioxane (150 mL) and H₂O (15 mL) was stirred for 2 hours at 80° C. under nitrogen atmosphere. The resulting mixture was concentrated under reduced pressure. The residue was purified by silica gel column chromatography, eluted with PE/EA (32%) to afford methyl 4-(5-isopropoxypyrimidin-2-yl)-5-methylthiophene-2-carboxylate (12.70 g, 67% yield) as a white solid. LCMS (ESI) [M+H]⁺: 293

Step 3: To a stirred solution of methyl 4-(5-isopropoxypyrimidin-2-yl)-5-methylthiophene-2-carboxylate (12.7 g, 43.4 mmol, 1 equiv) in EtOH (150 mL) and H₂O (150 mL) was added LiOH (10.4 g, 434.4 mmol, 10 equiv). Then the mixture was stirred for 4 hours at room temperature. The mixture was acidified to pH 5 with HCl (aq.). The precipitated solids were collected by filtration and washed with water. The resulting solid was dried under vacuum to afford 4-(5-isopropoxypyrimidin-2-yl)-5-methylthiophene-2-carboxylic acid (10.4 g, 86% yield) as a white solid. LCMS (ESI) [M+H]⁺: 279

Step 4: To a stirred solution of 4-(5-isopropoxypyrimidin-2-yl)-5-methylthiophene-2-carboxylic acid (10.4 g, 37.4 mmol, 1 equiv), TCFH (20.97 g, 74.7 mmol, 2 equiv) and NMI (12.27 g, 149.5 mmol, 4 equiv) in ACN (180 mL) was added N-(3-amino-5-chlorophenyl)methanesulfonamide (8.25 g, 37.4 mmol, 1 equiv). Then the mixture was stirred for 2 hours at room temperature. The mixture was concentrated and purified by reverse phase flash chromatography eluting with ACN/H₂O (56%, 0.1% FA) to afford N-(3-chloro-5-(methylsulfonamido)phenyl)-4-(5-isopropoxypyrimidin-2-yl)-5-methylthiophene-2-carboxamide (10.0113 g, 55.7% yield) as a white solid. LCMS (ESI) [M+H]⁺: 481. ¹H NMR (400 MHz, DMSO-d₆) δ 10.57 (s, 1H), 10.06 (s, 1H), 8.64 (d, J=2.7 Hz, 3H), 7.69 (dt, J=6.5, 1.9 Hz, 2H), 6.95 (t, J=2.0 Hz, 1H), 4.88 (p, J=6.0 Hz, 1H), 3.08 (s, 3H), 2.83 (s, 3H), 1.34 (d, J=6.0 Hz, 6H).

### Compound 4: 4-(5-(tert-butoxy)pyrimidin-2-yl)-N-(3-chloro-5-(methylsulfonamido)phenyl)-5-methylthiophene-2-carboxamide

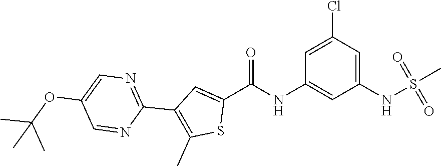 [SMILES: Cc1sc(C(=O)Nc2cc(Cl)cc(NS(C)(=O)=O)c2)cc1-c1ncc(OC(C)(C)C)cn1]

4-(5-(tert-butoxy)pyrimidin-2-yl)-N-(3-chloro-5-(methylsulfonamido)phenyl)-5-methylthiophene-2-carboxamide was prepared using a procedure similar to that described for Compounds 2 and 3. LCMS (ESI) [M+H]⁺: 495. ¹H NMR (400 MHz, DMSO-d₆) δ 10.57 (s, 1H), 10.07 (s, 1H), 8.66 (d, J=13.9 Hz, 3H), 7.69 (dt, J=8.3, 1.8 Hz, 2H), 6.95 (t, J=1.9 Hz, 1H), 3.08 (s, 3H), 2.86 (s, 3H), 1.38 (s, 9H).

### Compound 7: 4-bromo-N-(3-methanesulfonamidophenyl)thiophene-2-carboxamide

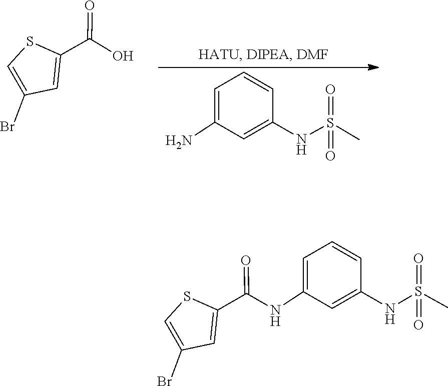 [SMILES: O=C(O)c1cc(Br)cs1 ; CS(=O)(=O)Nc1cccc(N)c1 ; CS(=O)(=O)Nc1cccc(NC(=O)c2cc(Br)cs2)c1]

To a solution of 4-bromothiophene-2-carboxylic acid (100 mg, 482 μmol), HATU (274 mg, 722 μmol) and N,N-diisopropylethylamine (185 mg, 1.44 mmol) in N,N-dimethylformamide (1.5 mL) was added N-(3-aminophenyl)methanesulfonamide (89.7 mg, 482 μmol). The mixture was stirred at room temperature overnight. The residue was purified by preparative HPLC to afford 4-bromo-N-(3-methanesulfonamidophenyl)thiophene-2-carboxamide (100 mg, 266 μmol).

¹H NMR (400 MHz, DMSO-d6) δ 10.36 (s, 1H), 9.84 (s, 1H), 8.10 (d, J=1.4 Hz, 1H), 8.02 (d, J=1.4 Hz, 1H), 7.65 (t, J=2.0 Hz, 1H), 7.51 (dt, J=8.0, 1.3 Hz, 1H), 7.30 (t, J=8.1 Hz, 1H), 7.00-6.85 (m, 1H), 3.01 (s, 3H).

LCMS (ESI-MS): [M+1]=377.00

In other embodiments, the DHX9 inhibitor is an antisense oligonucleotide (a single-stranded deoxyribonucleotide) complementary to DHX9 mRNA. In some embodiments, the DHX9 inhibitor is an RNA interfering agent. In some embodiments, the DHX9 inhibitor is selected from the group consisting of small interfering RNA (siRNA), a small hairpin RNA (shRNA), a microRNA (miRNA), or a piwiRNA (piRNA).

As used herein, the terms “short interfering RNA” and “siRNA” are used interchangeably to refer to any RNA molecule shorter than about 50, 40, 35, 30, 25, 20, or fewer nucleotides capable of inducing silencing of a gene target based on complementarity. Silencing is typically mediated by an Argonaute protein and is typically initiated by a short RNA molecule trigger. Silencing can be post-transcriptional or transcriptional. The siRNA molecule may be completely or partially complementary to the gene or genes whose expression in reduced, and silencing may be effected with or without cleavage of an mRNA transcript.

As used herein, the terms “short-hairpin RNA” and “shRNA” are used interchangeably to refer to any single-stranded RNA molecule having two regions capable of base-pairing with one another separated by a region that does not participate in base pairing, the processing of which releases one or more siRNAs. shRNAs may be of cellular (endogenous) or artificial (exogenous) origin. Endogenous siRNA includes “piRNA,” which are any of a class of Piwi-interacting RNAs.

As used herein, the term “microRNA,” “mature microRNA” and “miRNA” are used interchangeably to refer to a non-coding RNA molecule that has a length in the range of 19 to 30 nucleotides that participates in gene regulation. In some embodiments, the miRNA can be derived from “pre-microRNA” or “pre-miRNA”. “Pre-microRNA” or “pre-miRNA” are used interchangeably to refer to any molecule, the processing of which releases a mature miRNA.

Furthermore, as will be appreciated by those skilled in the art, some miRNA can be characterized in terms of their capacities to affect gene regulation in a wide variety of organisms and in a wide variety of tissues. A single gene may be regulated by multiple miRNAs, and miRNA regulation may act synergistically. A single miRNA may also be capable of regulating multiple mRNAs, for example, some estimates have suggested a single human miRNA may regulate up to 100-200 genes. It has also been recently described that up to 30% of human genes are targets for miRNA regulation.

Method of identifying or verifying a DHX9 inhibitor are known in the art. For example, exemplary methods of identifying or verifying a DHX9 inhibitor are described in the Examples of the present disclosure. In a non-limiting example, a candidate molecule that potentially inhibits DHX9 by inhibiting its expression can be verified by determining whether the candidate molecule inhibits or reduces expression of DHX9 mRNA or polypeptide in a cell. Methods for detecting mRNA levels such as northern blotting, in situ hybridization, ribonuclease protection assay (RPA) and RT-qPCR (see, e.g., Karen Reue, mRNA Quantitation Techniques: Considerations for Experimental Design and Application, The Journal of Nutrition, 1998, 128(11): 2038-2044; incorporated herein by reference), and methods for detecting polypeptide levels such as western blotting, ELISA, immunofluorescence staining, immunohistochemistry, and mass spectrometry, are well known in the art.

Alternatively, a candidate molecule that potentially inhibits DHX9, e.g., by inhibiting DHX9 function or activity of the polypeptide, can be verified by testing whether the candidate molecule inhibits ATPase activity of DHX9 (see, e.g., Example 2 of the present disclosure). Assays and kits for measuring ATPase activity of an ATP-dependent polypeptide such as DHX9 are known in the art and are commercially available.

In some embodiments, a candidate molecule that potentially inhibits DHX9, e.g., by inhibiting DHX9 function or activity of the polypeptide, can be verified by testing whether the candidate molecule inhibits or reduces a level of a product or a process that is dependent on DHX9 activity, such as RNA processing (see, e.g., Akta et al., DHX9 suppresses RNA processing defects originating from the Alu invasion of the human genome, Nature, 2017, 544:112-119; the entire contents of which are incorporated herein by reference).

Circular RNAs (circRNAs)

Multiple studies have reported that DHX9 binds to or binds near Alu elements more than other RNA-binding proteins (Akta et al., 2017; Stagsted et al. 2019, Noncoding AUG circRNAs constitute an abundant and conserved subclass of circles, Life Science Alliance, 2019, 2(3):1-16), incorporated herein by reference). Studies of DHX9 have also shown that DHX9 acts as a nuclear RNA resolvase, and resolves inverted-repeat Alu elements (IAEs) in order to de-repress translation of mRNAs with inverted-repeat Alu elements in their 3′ UTRs (Aktaş et al., 2017). It has been further reported that DHX9 inhibits circRNA production by unwinding and destabilizing RNA structures formed by inverted-repeat Alu elements (IAEs) in flanking regions of circRNAs (Stagsted et al.).

Suitable circRNAs for use in the methods and assays of the present disclosure are those circRNAs whose formation is modulated (e.g., reduced or prevented) or controlled by DHX9. In some embodiments a circRNA may be one whose formation is also modulated by mechanisms and/or molecules independent of DHX9, but is nevertheless useful in the methods and assays of the present disclosure as long as inhibition of DHX9 can induce an appreciable increase in formation of the circRNA.

In some embodiments, the circRNA useful in the methods and assays of the present disclosure is Alu-mediated circRNA. While not wishing to be bound by theory, it is believed that under normal conditions DHX9 binds to inverted-repeat Alu elements in flanking regions of mRNA and inhibits production of the circRNA. When DHX9 is inhibited (e.g., by using an siRNA to knock down DHX9 expression, or a small molecule DHX9 inhibitor to inhibit its activity), the inhibition of Alu-mediated circRNA production by DHX9 is reduced and the level of the Alu-mediated circRNA is increased.

Accordingly, circRNAs for use in the methods and assays of the present disclosure can be identified using methodologies described herein (see, e.g., the methods described in Example 9 of the present disclosure). For example, circRNAs, such as those known or predicted in databases (such as circBase (circbase.org) and CircNet (circnet.mbc.nctu.edu.tw)) or available for testing in commercial or customized arrays (e.g., Arraystar Human Circular RNA Array) can be tested for responsiveness to DHX9 inhibition. CircRNAs up-regulated following DHX9 inhibition are useful in the methods of the present disclosure. Such circRNAs can be compared to database or publications that annotate Alu elements (e.g., Stagsted et al. 2019) to arrive at Alu-mediated circRNAs that are DHX9-mediated. It is predicted that any circRNA identified as containing Alu elements (e.g., utilizing the foregoing databases) would be useful in the methods and assays of the present disclosure.

In some embodiments, the Alu-mediated circRNA is selected from the list of Alu-mediated circRNAs listed in Table 2 below. In some embodiments, more than one, e.g., 2, 3, 4, 5, 6, 7, 8, 9, 10 or more Alu-mediated circRNAs, e.g., selected from those listed in Table 2, are detected or measured in the methods of the present disclosure.

TABLE 2
List of Alu-mediated circRNAs (circRNAs)

| | | | | | Transcript | |
|---|---|---|---|---|---|---|
| Gene | | | | circRNA | Accession | |
| Symbol | circRNA | Alias | Source | type | No. | Sequence |
| AKR1A1 | hsa_circRNA_100204 | hsa_circ_0000067 | circBase | exonic | NM_006066 | CTGTACCTGATGCACTGGCCTTATGCCTTTGA |
| | | | | | | GTAAAAGCAGCTGTTAAGTATGCCCTTA |
| | | | | | | (SEQ ID NO: 25) |
| BMPR2 | hsa_circRNA_005078 | hsa_circ_0005078 | circBase | exonic | NM_001204 | GATCTTTCAGCCACAAATGTCCTGGATGGCA |
| | | | | | | GCAGTATACAGACAACCTGTCACATAATA |
| | | | | | | (SEQ ID NO: 26) |
| BRIP1 | hsa_circRNA_401855 | | 25242744 | exonic | NM_032043 | CGCCACAGAAATTAGGAATCTGAAATAAACA |
| | | | | | | GGAAAGCACTATGTCTTCAATGTGGTCTG |
| | | | | | | (SEQ ID NO: 27) |
| BRIP1 | hsa_circRNA_102149 | hsa_circ_0006968 | circBase | exonic | NM_032043 | CCAGACTGTGAGCCAAGGAATTTTGTGTTTCT |
| | | | | | | TGCCATCTTACAAGGCCTTTTCAGATAT |
| | | | | | | (SEQ ID NO: 28) |
| CALCOCO2 | hsa_circRNA_405600 | | 25070500 | exonic | NM_005831 | CTCAGAAAATGAGAAGATGGGAATCAGAGTG |
| | | | | | | GATCAGCTTCAGGTGGGGTGGAAGACAAC |
| | | | | | | (SEQ ID NO: 29) |
| CLNS1A | hsa_circRNA_100894 | hsa_circ_0000343 | circBase | exonic | NM_001293 | ACGATGGAGAAGAATATGATGTGGAAGCACA |
| | | | | | | TGAAGAATCAAAAGAACCTGTTGCTGATG |
| | | | | | | (SEQ ID NO: 30) |
| DKC1 | hsa_circRNA_092136 | hsa_circ_0092136 | circBase | exonic | NM_001363 | TCCTGAAAGAAGATTAGTAATTATTTTGCCAA |
| | | | | | | AGAAACATAAGAAGAAAAAGGAGCGGAA |
| | | | | | | (SEQ ID NO: 31) |
| FAM124A | hsa_circRNA_101264 | hsa_circ_0030292 | circBase | exonic | NM_145019 | CAGACGGAGGACCATGATGGGAACAAGATCC |
| | | | | | | TCCTACAGGTGAGCTTTCCGTTGAAGAGG |
| | | | | | | (SEQ ID NO: 32) |
| FBN1 | hsa_circRNA_035152 | hsa_circ_0035152 | circBase | exonic | NM_000138 | AAGGATACACAGGTGATGGCTTCACTTGTAC |
| | | | | | | AGATCCCATATGTGGTAAAGGGTACTCAA |
| | | | | | | (SEQ ID NO: 33) |
| FCHO2 | hsa_circRNA_103887 | hsa_circ_0002490 | circBase | exonic | NM_138782 | AATATCAGCAGACTAGAGCAGATTCTTCCAA |
| | | | | | | ATGCACAGCTTGTGTTCAGGTGTGTCACG |
| | | | | | | (SEQ ID NO: 34) |
| GLS | hsa_circRNA_102877 | hsa_circ_0057521 | circBase | exonic | NM_014905 | TGCTGGAGCAATTGTTGTGACTTCACTAATAA |
| | | | | | | AGATGTGTTCAGAGCAACATTGTTTTGT |
| | | | | | | (SEQ ID NO: 35) |
| GON4L | hsa_circRNA_100356 | hsa_circ_0014624 | circBase | exonic | NM_001282856 | AAATGTCATCAGCTTCTGAGGAATCTGTGCTT |
| | | | | | | TCTGTCCCAGAACTCCAGGCCAGTCTGC |
| | | | | | | (SEQ ID NO: 36) |
| GPR125 | hsa_circRNA_103610 | hsa_circ_0002785 | circBase | exonic | NM_145290 | AATAACCCAGACTAGGAATGCCAGCATCTGC |
| | | | | | | CAAGCAGATTTGACGGGATCTGAACTATA |
| | | | | | | (SEQ ID NO: 37) |
| NEIL3 | hsa_circRNA_005365 | hsa_circ_0005365 | circBase | exonic | NM_018248 | GCTGCAACCCTGGATATTCTAACAGGTTTGTC |
| | | | | | | AATTAACAGATGAACAGATCCATCACCT |
| | | | | | | (SEQ ID NO: 38) |
| NEIL3 | hsa_circRNA_403266 | | 25242744 | exonic | NM_018248 | ACCAGAGCTTAAAAGCTGCAACCCTGGATAT |
| | | | | | | TCTAACAGCAAGCTACCGACTAGAAATAC |
| | | | | | | (SEQ ID NO: 39) |
| PIK3C3 | hsa_circRNA_102355 | hsa_circ_0006527 | circBase | exonic | NM_002647 | TGCTAACAAAAACAGAACTTTTTTAGAAAAT |
| | | | | | | ATGCACCAAGTGAGAATGGGCCAAATGGG |
| | | | | | | (SEQ ID NO: 40) |
| PNN | hsa_circRNA_405210 | | 25070500 | exonic | NM_002687 | AATAGAAGAGTCACAGAGAAAAATGAACGC |
| | | | | | | AAAAGCGGCGCCAGGAAATTGAACAAAAAC |
| | | | | | | (SEQ ID NO: 41) |
| POMT1 | hsa_circRNA_104948 | hsa_circ_0001897 | circBase | exonic | NM_007171 | ATTTAGGAGGATTCGATGGCAATTTTTTGTGG |
| | | | | | | AACAGAATTGGAGCAGTTTTGACGAAGT |
| | | | | | | (SEQ ID NO: 42) |
| RBM23 | hsa_circRNA_101319 | hsa_circ_0000524 | circBase | exonic | NM_018107 | GAGACAAGCAAATCTGACAGGATGGCATCTG |
| | | | | | | ATGACTTTGACATAGTGATTGAGGCCATG |
| | | | | | | (SEQ ID NO: 43) |
| SKIL | hsa_circRNA_067938 | hsa_circ_0067938 | circBase | exonic | NM_005414 | TGAAGGAGAAGTTTAGCATGAGAAGTGGAAA |
| | | | | | | GAGAAATCAATCCAAGATTAATTAAAAGA |
| | | | | | | (SEQ ID NO: 44) |
| SLK | hsa_circRNA_000259 | hsa_circ_0000259 | circBase | exonic | NM_014720 | AGACATCAGCTACTTAAGCGCCACGAGAAGA |
| | | | | | | GTAAAAAGCGACAATATGACCAGGAAATT |
| | | | | | | (SEQ ID NO: 45) |
| SMA | hsa_circRNA_103868 | hsa_circ_0072797 | circBase | exonic | uc010ixi.1 | AAAGCAGGTCTTTTCTACACAGGGAGAACAT |
| | | | | | | TTTGTGAATTCCTGGGTCCAGAGAGAATT |
| | | | | | | (SEQ ID NO: 46) |
| SMG1 | hsa_circRNA_101736 | hsa_circ_0008216 | circBase | exonic | NM_015092 | CCTGATGAATGGTGGAGAGTTACGCAGATAA |
| | | | | | | TGCTAAGTTTGCAACAGAGAGCACAGAAA |
| | | | | | | (SEQ ID NO: 47) |
| SMYD4 | hsa_circRNA_405522 | | 25070500 | exonic | NM_052928 | CATACAACACACAGGGAGTGTCACATTCAAG |
| | | | | | | GCCTAACACTGAGGACATGTCACTGTGTC |
| | | | | | | (SEQ ID NO:48) |
| TAF2 | hsa_circRNA_002593 | hsa_circ_0002593 | circBase | intronic | ENST00000521007 | TCAGGGAACCCTGATTTACTCCTGTAACTTTC |
| | | | | | | CATTTCACAAAGGGATTTGTGGAACTGA |
| | | | | | | (SEQ ID NO: 49) |
| TFAP4 | hsa_circRNA_002439 | hsa_circ_0002439 | circBase | exonic | NM_003223 | GACAAGGAGCGCTCGGTGCGCATGATGCTGG |
| | | | | | | AGGAGCAGCCTTGCCAACATTCCACTAAC |
| | | | | | | (SEQ ID NO:50) |
| TMEM41B | hsa_circRNA_021157 | hsa_circ_0021157 | circBase | exonic | NM_015012 | GTCACAGCAGAGAAGAAAGAGTGAATATGA |
| | | | | | | AGGTTCCCAGAGATATGGATGATGCCAAGG |
| | | | | | | (SEQ ID NO: 51) |
| USP25 | hsa_circRNA_103113 | hsa_circ_0005238 | circBase | exonic | NM_013396 | GATAAAAGTCCTGAAACAGTTTTGCAGTCGA |
| | | | | | | GGAGTTTAATAAAGAAACTGGGCAGCCCC |
| | | | | | | (SEQ ID NO: 52) |
| VPS13D | hsa_circRNA_100055 | hsa_circ_0009964 | circBase | exonic | NM_015378 | CTTTGAATCTTTGCATAGAGGTCAAGATAACT |
| | | | | | | CCAGGAGGAAAAGTAGGGATGGGTCAGC |
| | | | | | | (SEQ ID NO: 53) |
| WDR59 | hsa_circRNA_004910 | hsa_circ_0004910 | circBase | exonic | NM_030581 | TGTGCAAATCCGGAATGTCAATGTGGAGCTT |
| | | | | | | TGTGCAAATGACATATTAGATGGTGTTGA |
| | | | | | | (SEQ ID NO: 54) |
| XPR1 | hsa_circRNA_100413 | hsa_circ_0005909 | circBase | exonic | NM_004736 | TGGCTGCTTAAACTGCTGCTGTATTTAAACTT |
| | | | | | | GAAACAGATAGAAGTATATGGCCCTTGA |
| | | | | | | (SEQ ID NO: 55) |
| XPR1 | hsa_circRNA_015513 | hsa_circ_0015513 | circBase | exonic | NM_004736 | TTTTGTGGAATATTCATTGTACTGAATATTAC |
| | | | | | | CCTTGTGCTTGCCGAGAAGCTCGCAGAG |
| | | | | | | (SEQ ID NO: 56) |

In some embodiments, the circRNA is derived from the BRIP1 gene, and called circBRIP1. BRIP1, also known as “BRCA1 Interacting Protein C-terminal helicase 1”, is a tumor suppressor gene interacting with another known DNA repair gene, BRCA-1 (Breast Cancer gene 1), involved in repair by homologous recombination. Mutations in the BRIP1 gene are known to increase the risk of ovarian and breast cancers. Human BRIP1 nucleotide and amino acid sequences can be found at GenBank Accession No. NM_032043.3, incorporated herein by reference.

In some embodiments, the circRNA is derived from the AKR1A1 gene. AKR1A1, also known as aldehyde reductase, is involved in the synthesis of ascorbic acid (AsA) as well as the detoxification of aldehydes. Human AKR1A1 nucleotide and amino acid sequences can be found at GenBank Accession No. NM_006066.4, NM_153326.3, NM_001202413.2, or NM_001202414.2, incorporated herein by reference.

In some embodiments, the circRNA is derived from the BMPR2 gene. The BMPR2 gene, also known as “bone morphogenetic protein receptor type 2”, belongs to a family of genes originally identified for its role in regulating the growth and maturation (differentiation) of bone and cartilage. Recently, researchers have found that this gene family plays a broader role in regulating the growth and differentiation of numerous types of cells. Human BMPR2 nucleotide and amino acid sequences can be found at GenBank Accession No. NM_001204.7, incorporated herein by reference.

In some embodiments, the circRNA is derived from the CALCOCO2 gene. CALCOCO2, also known as “calcium Binding And Coiled-Coil Domain 2”, is a receptor for ubiquitin-coated bacteria and plays an important role in innate immunity by mediating macroautophagy. Human CALCOCO2 nucleotide and amino acid sequences can be found at GenBank Accession No. NM_005831.5, NM_001261390.2, or NM_001261391.2, incorporated herein by reference.

In some embodiments, the circRNA is derived from the CLNS1A gene. CLNSIA, also known as “chloride nucleotide-sensitive channel 1A” functions in multiple regulatory pathways.

The encoded protein complexes with numerous cytosolic proteins and performs diverse functions including regulation of small nuclear ribonucleoprotein biosynthesis, platelet activation and cytoskeletal organization. The protein is also found associated with the plasma membrane where it functions as a chloride current regulator. Human CLNS1A nucleotide and amino acid sequences can be found at GenBank Accession No. NM_001293.3, NM_001311199.2, NM_001311200.2, NM_001311201.2, or NM_001311202.2, incorporated herein by reference.

In some embodiments, the circRNA is derived from the DKC1 gene. DKC1, also known as “dyskerin pseudouridine synthase 1” plays an active role in telomerase stabilization and maintenance, as well as recognition of snoRNAs containing H/ACA sequences which provides stability during biogenesis and assembly into H/ACA small nucleolar RNA ribonucleoproteins (snoRNPs). This gene is highly conserved and widely expressed, and may play additional roles in nucleo-cytoplasmic shuttling, DNA damage response, and cell adhesion. Human DKC1 nucleotide and amino acid sequences can be found at GenBank Accession No. NM_001363.5, NM_001142463.3, NM_001288747.2, NR_110021.2, NR_110022.2, or NR_110023.2, incorporated herein by reference.

In some embodiments, the circRNA is derived from the FAM124A gene. FAM124A, is also known as “family with sequence similarity 124 member A”. Human FAM124A nucleotide and amino acid sequences can be found at GenBank Accession No. NM_145019.4, NM_001242312.2, or NM_001330522.2, incorporated herein by reference.

In some embodiments, the circRNA is derived from the FBN1 gene. FBN1, also known as fibrillin 1, is proteolytically processed to generate two proteins including the extracellular matrix component fibrillin-1 and the protein hormone asprosin. Fibrillin-1 is an extracellular matrix glycoprotein that serves as a structural component of calcium-binding microfibrils. These microfibrils provide force-bearing structural support in elastic and nonelastic connective tissue throughout the body. Asprosin, secreted by white adipose tissue, has been shown to regulate glucose homeostasis. Human FBN1 nucleotide and amino acid sequences can be found at GenBank Accession No. NM_000138.5, NM_001406716.1, NM_001406717.1, or NM_001406718.1, incorporated herein by reference.

In some embodiments, the circRNA is derived from the FCHO2 gene. FCHO2, also known as, “FCH and mu domain containing endocytic adaptor 2”, functions in an early step of clathrin-mediated endocytosis. Human FCHO2 nucleotide and amino acid sequences can be found at GenBank Accession No. NM_138782.3 or NM_001146032.2, incorporated herein by reference.

In some embodiments, the circRNA is derived from the GLS gene. GLS, also known as glutaminase, an phosphate-activated amidohydrolase that catalyzes the hydrolysis of glutamine to glutamate and ammonia. Human GLS nucleotide and amino acid sequences can be found at GenBank Accession No. NM_014905.5 or NM_001256310.2, incorporated herein by reference.

In some embodiments, the circRNA is derived from the GON4L gene. GON4L, also known as “GON-4 Like”, is a nuclear protein containing two serine phosphosites and a lysine-glutamine cross-link and is thought to be a transcription factor. Human GON4L nucleotide and amino acid sequences can be found at GenBank Accession No. NM_001282856.2, NM_032292.6, NM_001282860.2, NM_001282861.2, or NM_001282858.2, incorporated herein by reference.

In some embodiments, the circRNA is derived from the GPR125 gene. GPR125, also known as “adhesion G protein-coupled receptor A3” or “ADGRA3”, is a member of the G protein-coupled receptor superfamily. This membrane protein may play a role in tumor angiogenesis through its interaction with the human homolog of the Drosophila disc large tumor suppressor gene. Human GPR125 nucleotide and amino acid sequences can be found at GenBank Accession No. NM_145290.4, incorporated herein by reference.

In some embodiments, the circRNA is derived from the NEIL3 gene. Neil3, also known as “nei like DNA glycosylase 3”, belongs to a class of DNA glycosylases. These glycosylases initiate the first step in base excision repair by cleaving bases damaged by reactive oxygen species and introducing a DNA strand break via the associated lyase reaction Human NEIL3 nucleotide and amino acid sequences can be found at GenBank Accession No. NM_018248.3, incorporated herein by reference.

In some embodiments, the circRNA is derived from the PIK3C3 gene. PIK3C3, also known as “phosphatidylinositol 3-kinase catalytic subunit type 3”, belongs to the phosphoinositide 3-kinase (PI3K) family. PIK3C3 can phosphorylate phosphatidylinositol (PtdIns) to generate phosphatidylinositol 3-phosphate (PI3P), a phospholipid central to autophagy. Inhibition of PIK3C3 successfully inhibits autophagy. Human PIK3C3 nucleotide and amino acid sequences can be found at GenBank Accession No. NM_002647.4 or NM_001308020.2, incorporated herein by reference.

In some embodiments, the circRNA is derived from the PNN gene. PNN, also known as “pinin, desmosome associated protein”, is a nuclear and cell adhesion-related protein. Studies have suggested that Pnn/DRS/memA is a potential tumor suppressor involved in the regulation of cell adhesion and cell migration. Human PNN nucleotide and amino acid sequences can be found at GenBank Accession No. NM_002687.4, incorporated herein by reference.

In some embodiments, the circRNA is derived from the POMT1 gene. POMT1, also known as “protein O-mannosyltransferase 1”, is a key enzyme in the glycosylation of α-dystroglycan. Human POMT1 nucleotide and amino acid sequences can be found at GenBank Accession No. NM_007171.4, NM_001077365.2, or NM_001077366.2, incorporated herein by reference.

In some embodiments, the circRNA is derived from the RBM23 gene. RMB23, also known as “RNA binding motif protein 23”, is a member of the U2AF-like family of RNA binding proteins. This protein interacts with some steroid nuclear receptors, localizes to the promoter of a steroid-responsive gene, and increases transcription of steroid-responsive transcriptional reporters in a hormone-dependent manner. Human RBM23 nucleotide and amino acid sequences can be found at GenBank Accession No. NM_018107.5, NM_001077351.2, NM_001077352.2, NM_001308044.2, NM_001352762.2, NM_001352763.2, NM_001352764.2, NM_001352765.2 or NM_001352766.2, incorporated herein by reference.

In some embodiments, the circRNA is derived from the SKIL gene. SKIL, also known as “SKI like proto-oncogene”, is a component of the SMAD pathway, which regulates cell growth and differentiation through transforming growth factor-beta (TGFB). In the absence of ligand, the encoded protein binds to the promoter region of TGFB-responsive genes and recruits a nuclear repressor complex. TGFB signaling causes SMAD3 to enter the nucleus and degrade this protein, allowing these genes to be activated. Human SKIL nucleotide and amino acid sequences can be found at GenBank Accession No. NM_005414.5, NM_001145097.2, NM_001145098.3, or NM_001248008.1, incorporated herein by reference.

In some embodiments, the circRNA is derived from the SLK gene. Human SLK nucleotide and amino acid sequences can be found at GenBank Accession No. NM_014720.4 or NM_001304743.2, incorporated herein by reference.

In some embodiments, the circRNA is derived from the SMA gene. SMA, also known as “survival of motor neuron 1”, is part of a 500 kb inverted duplication on chromosome 5q13. This duplicated region contains at least four genes and repetitive elements which make it prone to rearrangements and deletions. The protein encoded by this gene localizes to both the cytoplasm and the nucleus. Within the nucleus, the protein localizes to subnuclear bodies called gems which are found near coiled bodies containing high concentrations of small ribonucleoproteins (snRNPs). This protein forms heteromeric complexes with proteins such as SIP1 and GEMIN4, and also interacts with several proteins known to be involved in the biogenesis of snRNPs, such as hnRNP U protein and the small nucleolar RNA binding protein. Human SMA nucleotide and amino acid sequences can be found at GenBank Accession No. NM_001297715.1, NM_022874.2, or NM_000344.4, incorporated herein by reference.

In some embodiments, the circRNA is derived from the SMG1 gene. SMG1, also known as “SMG1 nonsense mediated mRNA decay associated PI3K related kinase”, is involved in nonsense-mediated mRNA decay (NMD) as part of the mRNA surveillance complex. The protein has kinase activity and is thought to function in NMD by phosphorylating the regulator of nonsense transcripts 1 protein. Human SMG1 nucleotide and amino acid sequences can be found at GenBank Accession No. NM_015092.5, incorporated herein by reference.

In some embodiments, the circRNA is derived from the SMYD4 gene. SMYD4, also known as “SET and MYND domain containing 4”, is a potential tumor suppressor Human SMYD4 nucleotide and amino acid sequences can be found at GenBank Accession No. NM_052928.4, incorporated herein by reference.

In some embodiments, the circRNA is derived from the TAF2 gene. TAF2 (TATA-box binding protein associated factor 2) encodes one of the larger subunits of transcription factor IID (TFIID) that is stably associated with the TFIID complex. It contributes to interactions at and downstream of the transcription initiation site, interactions that help determine transcription complex response to activators. Human TAF2 nucleotide and amino acid sequences can be found at GenBank Accession No. NM_003184.4, incorporated herein by reference.

In some embodiments, the circRNA is derived from the TFAP4 gene. TFAP4, also known as “transcription factor AP-4”, is a transcription factor of the basic helix-loop-helix-zipper (bHLH-ZIP) family contain a basic domain, which is used for DNA binding, and HLH and ZIP domains, which are used for oligomerization. Transcription factor AP4 activates both viral and cellular genes by binding to the symmetrical DNA sequence CAGCTG. Human TFAP4 nucleotide and amino acid sequences can be found at GenBank Accession No. NM_003223.3, incorporated herein by reference.

In some embodiments, the circRNA is derived from the TMEM41B gene. TMEM41B, also known as “transmembrane protein 41B”, is an integral endoplasmic reticulum (ER) membrane protein distantly that plays roles in autophagosome biogenesis. Human TMEM4IB nucleotide and amino acid sequences can be found at GenBank Accession No. NM_015012.4, incorporated herein by reference.

In some embodiments, the circRNA is derived from the USP25 gene. USP25, also known as “ubiquitin specific peptidase 25”, is a deubiquitinating enzyme. Human USP25 nucleotide and amino acid sequences can be found at GenBank Accession No. NM_013396.6, NM_001283041.3, NM_001283042.3, NM_001352560.2, NM_001352561.2, NM_001388299.1, NM_001388300.1, NM_001388301.1 or, NM_001388302.1, incorporated herein by reference.

In some embodiments, the circRNA is derived from the VPS13D gene. VPS13D, also known as “vacuolar protein sorting 13 homolog D”, is a protein belonging to the vacuolar-protein-sorting-13 gene family. In yeast, vacuolar-protein-sorting-13 proteins are involved in trafficking of membrane proteins between the trans-Golgi network and the prevacuolar compartment. Human VPS13D nucleotide and amino acid sequences can be found at GenBank Accession No. NM_015378.4 or NM_018156.4, incorporated herein by reference.

In some embodiments, the circRNA is derived from the WDR59 gene. WDR59, also known as “WD repeat domain 59”, is involved in cellular response to amino acid starvation and positive regulation of TOR signaling. Human WDR59 nucleotide and amino acid sequences can be found at GenBank Accession No. NM_030581.4, NM_001324171.2, or NM_001324172.2, incorporated herein by reference.

In some embodiments, the circRNA is derived from the XPR1 gene. XPR1, also known as “xenotropic and polytropic retrovirus receptor 1”, is a receptor for the xenotropic and polytropic classes of murine leukemia viruses. The encoded protein is involved in phosphate homeostasis by mediating phosphate export from the cell. Human XPR1 nucleotide and amino acid sequences can be found at GenBank Accession No. NM_004736.4, NM_001135669.2, NM_001328662.2, or NR_137330.2, incorporated herein by reference.

Methods of Detecting circRNAs

CircRNA can be detected and measured using methods commonly known in the art. For example, in some embodiments, circRNA is detected and/or measured by employing a technique selected from the group consisting of northernblot, reverse transcription-quantitative polymerase chain reaction (RT-qPCR), microarray, droplet digital PCR, next-generation sequence (NGS) RNA sequencing, RNA in situ hybridization (RNA-ISH), in situ hybridization-immunohistochemistry (ISH-IHC), isothermal exponential amplification and rolling cycle amplification. In some embodiments, circRNA can be detected using quantitative real-time PCR, e.g., Taqman PCR, e.g., Taqman Multiplex PCR. In some embodiments, circRNA can be detected using SYBR Green PCR. Additional methods of detecting circRNA are known in the art, and can be found at, for example, in Mi et al., Circular RNA detection methods: A minireview, Talanta, 2022, 238(2): 123066, the entire contents of which are incorporated herein by reference.

In some embodiments, circRNA is detected by RT-qPCR. In some embodiments, the circRNA is a circBRIP1, and the circBRIP1 is detected by RT-qPCR using the primers as described in any of Examples 1-9 of the present disclosure.

In some embodiments, the forward and reverse primers used to detect circBRIP are TCTGTGTGCCAGACTGTGAG (SEQ ID NO: 9) and ACACCAAGTTCTGACGAAAAGG (SEQ ID NO: 10), respectively.

In some embodiments, the forward and reverse primers used to detect circBRIP are GTGAGCCAAGGAATTTTGTGTTT (SEQ ID NO: 19) and GGTCTGAACTTTGCCATTAATATCTG (SEQ ID NO: 20), respectively.

In some embodiments, the forward and reverse primers used to detect circBRIP are TCAAAATAGAAGCCAGTGGATGAG (SEQ ID NO: 22) and AATGGCATGCACAAGAACATG (SEQ ID NO: 23), respectively.

Levels of circRNA markers can be detected based on the absolute level or a normalized or relative level. Detection of absolute circRNA levels may be preferable when monitoring the treatment of a cell or subject. For example, the level of one or more circRNA markers (e.g., circBRIP1) can be monitored in a cell or a subject receiving a DHX9 inhibitor, e.g., at regular intervals, such as daily or weekly intervals. A modulation in the level of one or more circRNA markers can be monitored over time to observe trends in changes in marker levels. Expression levels of the markers of the invention, e.g., circBRIP1, in the subject may be higher than the expression level of those markers in a prior expression level (e.g., prior to treatment, or from an earlier time point), thus indicating engagement of the DHX9 target by the inhibitor, inhibition of DHX9 by the inhibitor, or responsiveness or potential therapeutic effects of the treatment regimen for the subject.

As an alternative to making determinations based on the absolute expression level of the circRNA marker, determinations may be based on the normalized level of the marker. Levels are normalized by correcting the absolute level of a marker by comparing its level to the expression of a gene that is not a marker, e.g., a housekeeping gene that is constitutively expressed. Suitable genes for normalization include housekeeping genes such as the actin gene, or GAPDH gene.

This normalization allows the comparison of the level in one sample to another sample (e.g., comparison between samples from cells or subjects receiving different amount of the same inhibitor, or comparison between samples from cells or subjects receiving different inhibitors).

### EXAMPLES

### Example 1: Probing circRNAs after DHX9 Inhibition In Vitro

This experiment was conducted to evaluate circular and linear formats of different mRNA in HCT116 cells after treatment with DHX9 inhibitors by Quantitative Polymerase Chain Reaction (qPCR).

### Methods

HCT116 cells were obtained from ATCC (CCL-247) and were grown and assayed in McCoy's 5a growth media supplemented with 10% FBS.

The cells were plated at pre-determined cell densities in 384-well cell culture plates and incubated overnight in 37° C. at 5% CO₂. In a separate plate, reference and test compounds were prepared in DMSO stock solution and serially diluted 3-fold for 10 points. Compound 7 (0.1% final DMSO concentration) was transferred to the cell plate in designated wells, and incubated 37° C. at 5% CO₂ for 72 hours.

RNA was extracted from the cells using the SYBR Green Fast Advanced Cell-to-CT kit according to manufacturer's (Invitrogen) protocol, and cDNA was reverse transcribed using the High Capacity RNA-to-cDNA Kit according to the manufacturer's (Invitrogen) protocol. Using RT² SYBR Green ROX qPCR Mastermix (Invitrogen) and thermal cycler conditions for the QuantStudio Thermocycler, qPCR was carried out using the below custom primer sequences obtained at Integrated DNA Technologies (IDT):

TABLE 3
DNA Oligo Primers
DNA Oligo Primers

| | Circular (Circ) | Forward (F) | | SEQ ID |
|---|---|---|---|---|
| Gene | or Linear (Lin) | or Reverse (R) | Sequence | NO: |
| AKR1A1 | Circ | F | GAA GAC TCT GGC TGA CCT CC | 1 |
| AKR1A1 | Circ | R | AGC CTA CGC TAA GGG CAT AC | 2 |
| AKR1A1 | Lin | F | AGG TCC AGC GGA AAG TGA TC | 3 |
| AKR1A1 | Lin | R | TGT TCA GGG CAT TTA GCT GC | 4 |
| DKC1 | Circ | F | GGA TGA GAG TTA CCTGCG GC | 5 |
| DKC1 | Circ | R | AAT GTA GGT GCC AGC CTC AC | 6 |
| DKC1 | Lin | F | ATG AGG ACG GCA TTG AGG TC | 7 |
| DKC1 | Lin | R | GTC GCA GGT AGA GAT GAC CG | 8 |
| BRIP1 | Circ | F | TCT GTG TGC CAG ACT GTG AG | 9 |
| BRIP1 | Circ | R | ACA CCA AGT TCT GAC GAA AAG G | 10 |
| BRIP1 | Lin | F | GTC CAT CCT GAG GTA GTC GG | 11 |
| BRIP1 | Lin | R | TTC CCC AGG CTG ACA AGT TC | 12 |
| SETD3 | Circ | F | TGAAGAAGATGAAGTTCGGTAT | 13 |
| SETD3 | Circ | R | GTGCCAGATTTCTGAGTTTT | 14 |
| SETD3 | Lin | F | GACAGACTCTACGCCATG | 15 |
| SETD3 | Lin | R | GTCTCCCAGCAAGTGTTC | 16 |
| GAPDH | Lin | F | GTCTCCTCTGACTTCAACAGCG | 17 |
| GAPDH | Lin | R | ACCACCCTGTTGCTGTAGCCAA | 18 |

Note:
Forward and Reverse primers are combined per gene, and each sample has to be run on a separate plate for each gene.

Data analysis was performed by using the QuantStudio Thermocycler software to determine threshold Ct values. ΔCt for circRNA or linRNA is calculated by normalizing the target gene Ct to the GAPDH housekeeping gene Ct for each sample. The inhibition activity is calculated by the following formula, whereas:

ΔCts: Mean of Ct(target gene)−Mean of Ct(GAPDH) for each sample

ΔCtc: Mean of Ct(target gene)−Mean of Ct(GAPDH) for DMSO control.

$$\% Vehicle = 100 \% \times \frac{{2}^{Δ Cts}}{{2}^{Δ Ctc}}$$

### Results

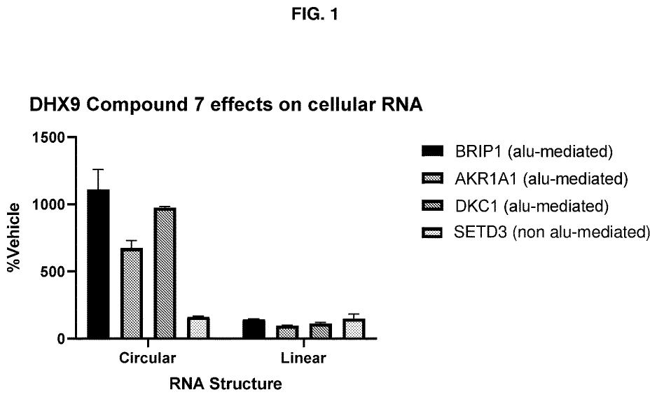

CirRNAs of BRIP1, AKR1A1 and DKC1 are known to be Alu-mediated, and cirRNA of SETD3 is non-Alu-mediated. Upon DHX9 inhibition by Compound 7, the Alu-mediated circRNAs, namely BRIP1, AKR1A1 and DKC1, were induced, while no induction of SETD3 cirRNA was observed (see FIG. 1).

### Example 2: Correlations of circBRIP1 with Anti-Tumor Activity of DHX9 Inhibitors In Vitro

This study was conducted to evaluate the circular format of BRIP1 in response to treatment with a panel of DHX9 inhibitors in vitro.

### Methods

DHX9 Inhibitor compounds: A panel of DHX9 inhibitor compounds were used in this Example. The panel includes Compounds 1, 2, 3, 4 as shown in Table 1 and compounds which have been described in International Application No. PCT/US2023/012929, the entire contents of which are expressly incorporated by reference herein.

ATPase Assay: DHX9 ATPase assay was performed in small-volume, nonbinding, 384-well white plates at a final volume of 10 μL/well. A panel of test compounds (10 mM solution in DMSO; 100 nL/well) were serially diluted on Bravo (Agilent, Santa Clara, CA) and dispensed into wells of columns 3-22 of the plates using an Echo 555 acoustic dispenser (Labcyte, Sunnyvale, CA). 100 nL of Aurintricarboxylic acid was dispensed into low control wells and 100 nL of DMSO was dispensed into high control wells. Then, a Multidrop Combi Reagent Dispenser (Thermo Fisher Scientific, Waltham, MA) was used to add a solution of DHX9 (1.25 nM, 5 μL/well) in assay buffer (40 mM HEPES [pH 7.5], 0.01% Tween 20, 0.01% BSA, 1 mM DTT, 5 mM MgCl₂, 0.004 U/ml RNAseOUT). The reaction was initiated by the addition of 5 μL of substrate solution (30 nM double-stranded RNA substrate, 10 μM Ultra Pure ATP in assay buffer) into the wells. The plates were incubated at room temperature for 1 h. After the indicated incubation times, 10 μL ADP-Glo reagent was added to the reactions and the plate was incubated at room temperature for 40 min. Then, 20 μL of kinase detection reagent was added and after an incubation time of 40 min, luminescence was recorded on Envision plate reader (Perkin-Elmer, Billerica, MA). All data was analyzed by Accent Therapeutics internal data analysis software developed by Scigilian (Montreal, Canada). The percentage inhibition was calculated based on the high control (DMSO) as 0% inhibition, and low control (10 μM Aurintricarboxylic acid) control as 100% inhibition and used for the calculation of IC₅₀ and IP (inflection point) values by fitting the dose-response curves to a four-parameter logistic model. Assay results reported IP values instead of IC₅₀ values since several compounds did not reach 100% inhibition at higher compound concentrations.

Cellular Proliferation Assay: The CellTiter-Glo® Luminescent Cell Viability Assay is a homogeneous method to determine the number of viable cells in culture based on quantitation of the ATP present, which signals the presence of metabolically active cells. LS411N cells (CRL-2159), obtained from ATCC, were grown in RPMI-1640 media supplemented with 10% FBS.

The cells were plated at pre-determined cell densities in 384-well solid white cell culture plates and incubated overnight in 37° C. at 5% CO₂. In a separate plate, reference and a panel of test compounds were prepared in DMSO stock solution and serially diluted 3-fold for 10 points. Compound (0.1% final DMSO concentration) was transferred to the cell plate in designated wells, and incubated 37° C. at 5% CO₂ for 120 hours. CellTiter-Glo reagent was prepared fresh according to manufacturer's (Promega) directions and added to each well. The plate was then shaken at 300 rpm for 10 minutes at RT, and then read on an Envision plate reader using a Luminescence protocol.

Data analysis was performed by normalizing the raw data (raw luminescence units or RSample) to an average of the positive control values for wells containing culture media only (100% cell death or RLC) and the negative control values for 0.1% DMSO (0% cell death or RHC). An IC₅₀ was calculated using a 4-parameter logistic nonlinear regression model in Scigilian Analyzer or GraphPad Prism, constraining the top parameter to 100 and the bottom parameter to 0.

$$\% Inhibition = 100 \% \times ( 1 - \frac{{R}_{sample} - {R}_{LC}}{{R}_{HC} - {R}_{LC}} )$$

$${IC}_{50} Fit formula = ( A + ( ( B - A ) / ( 1 + ( ( x / C ) \hat{} D ) ) ) ) …$$

$$whereas A : Bottom ; B : Top ; C : {IC}_{50} ; D : Slope$$

circBRIP1 Cellular Target Engagement Taqman Multiplex Assay: Evaluation of the circular format of BRIP1 mRNA in HCT116 cells by Quantitative Polymerase Chain Reaction (qPCR) using the Taqman Multiplex assay.

HCT116 cells were obtained from ATCC (CCL-247) and were grown and assayed in McCoy's 5a growth media supplemented with 10% FBS. The cells were plated at pre-determined cell densities in 384-well cell culture plates and incubated overnight in 37° C. at 5% CO₂. In a separate plate, reference and a panel of test compounds were prepared in DMSO stock solution and serially diluted 3-fold for 10 points. Compound (0.1% final DMSO concentration) was transferred to the cell plate in designated wells, and incubated 37° C. at 5% C02 for 72 hours.

RNA was extracted from the cells using the TaqMan Gene Expression Cells-to-CT™ kit according to manufacturer's (Invitrogen) protocol, and cDNA was reverse transcribed using the High Capacity RNA-to-cDNA Kit according to the manufacturer's (Invitrogen) protocol. Using TaqMan Multiplex Mastermix and thermal cycler conditions for the QuantStudio Thermocycler, qPCR was carried out using TaqMan GAPDH primer limited assay with JUN dye/QSY probe, and the below custom TaqMan Gene Expression Assay with FAM Dye for circBRIP1:

•Forward Primer:
(SEQ ID NO: 19)
GTGAGCCAAGGAATTTTGTGTTT
•Reverse Primer:
(SEQ ID NO: 20)
GGTCTGAACTTTGCCATTAATATCTG
•Probe:
(SEQ ID NO: 21)
CCATCTTACAAGGCCTTT

Note: All target genes can be combined in one well per sample.

Data analysis was performed by using the QuantStudio Thermocycler software to determine threshold Ct values. ΔCt for circBRIP1 or linBRIP1 is calculated by normalizing the target gene Ct to the GAPDH housekeeping gene Ct for each sample. The inhibition activity is calculated by the following formula, whereas:

ΔCts: Mean of Ct(target gene)−Mean of Ct(GAPDH) for each sample

ΔCtc: Mean of Ct(target gene)−Mean of Ct(GAPDH) for DMSO control.

$$\% Inhibition = 100 \% \times \frac{{2}^{Δ Cts}}{{2}^{Δ Ctc}}$$

An EC₅₀ was then calculated using a 4-parameter logistic nonlinear regression model in Scigilian Analyzer or GraphPad Prism, floating both the top and bottom parameters.

IC₅₀ Fit formula=(A+((B−A)/(1+((x/C)\hat{ }D)))) . . . whereas A:Bottom; B:Top; C:IC50; D:Slope

circBRIP1 Cellular Target Engagement SYBR Green Assay:

The circular format of BRIP1 mRNA in HCT116 cells was additionally evaluated by Quantitative Polymerase Chain Reaction (qPCR) using the SYBR Green assay.

HCT116 cells were obtained from ATCC (CCL-247) and were grown and assayed in McCoy's 5a growth media supplemented with 10% FBS. The cells were plated at pre-determined cell densities in 384-well cell culture plates and incubated overnight in 37° C. at 5% CO₂. In a separate plate, reference and test compounds were prepared in DMSO stock solution and serially diluted 3-fold for 10 points. Compound 7 (0.1% final DMSO concentration) was transferred to the cell plate in designated wells, and incubated 37° C. at 5% CO₂ for 72 hours.

RNA was extracted from the cells using the SYBR Green Fast Advanced Cell-to-CT kit according to manufacturer's (Invitrogen) protocol, and cDNA was reverse transcribed using the High Capacity RNA-to-cDNA Kit according to the manufacturer's (Invitrogen) protocol. Using RT² SYBR Green ROX qPCR Mastermix (Invitrogen) and thermal cycler conditions for the QuantStudio Thermocycler, qPCR was carried out using the below custom primer sequences:

•BRIP1-Circular-Forward
(SEQ ID NO: 9)
(TCT GTG TGC CAG ACT GTG AG)
•BRIP1-Circular-Reverse
(SEQ ID NO: 10)
(ACA CCA AGT TCT GAC GAA AAG G)
•GAPDH-Forward
(SEQ ID NO: 17)
(GTCTCCTCTGACTTCAACAGCG)
•GAPDH-Reverse
(SEQ ID NO: 18)
(ACCACCCTGTTGCTGTAGCCAA)

Note: Forward and Reverse primers are combined per gene, and each sample has to be run on a separate plate for each gene.

Data analysis was performed by using the QuantStudio Thermocycler software to determine threshold Ct values. ΔCt for circRNA is calculated by normalizing the target gene Ct to the GAPDH housekeeping gene Ct for each sample. The inhibition activity is calculated by the following formula, whereas:

ΔCts: Mean of Ct(target gene)−Mean of Ct(GAPDH) for each sample

ΔCtc: Mean of Ct(target gene)−Mean of Ct(GAPDH) for DMSO control.

$$\% Vehicle = 100 \% \times \frac{{2}^{Δ Cts}}{{2}^{Δ Ctc}}$$

### Results

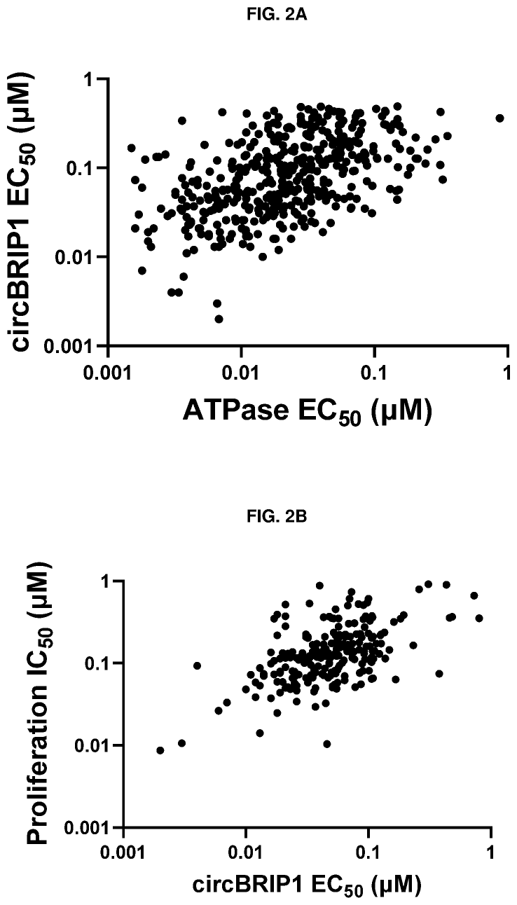

The results of this study, shown in FIGS. 2A-2B, indicate that cellular target engagement by the DHX9 inhibitors and the resulting induction of circBRIP1 correlates with the inhibition of DHX9 ATPase activity and anti-proliferative activity of DHX9 inhibitors in certain MSI cancers.

### Example 3: Evaluation of circBRIP1 in Human Xenograft Tumor

This study was conducted to evaluate the circular and linear format of human BRIP1 mRNA in LS411N xenograft tumors by qPCR.

### Methods

Experiments were performed in female BALB/c nude mice (GenPharmatech Co.). Animals were allowed to acclimate for 7 days before the study. The general health of the animals were evaluated by a veterinarian, and complete health checks were performed prior to the study. General procedures for animal care and housing were in accordance with the standard, Commission on Life Sciences, National Research Council, Standard Operating Procedures (SOPs) of Pharmaron, Inc. The mice were kept in laminar flow rooms at constant temperature and humidity with 3-5 mice in each cage. Animals were housed in polycarbonate cage which is the size of 300×180×150 mm³ and in an environmentally monitored, well-ventilated room maintained at a temperature of (23±3° C.) and a relative humidity of 40% 70%. Fluorescent lighting provided illumination approximately 12 hours per day. Animals had free access to irradiation sterilized dry granule food during the entire study period except for time periods specified by the protocol, as well as sterile drinking water in a bottle was available ad libitum during the quarantine and study periods.

The LS411N (ATCC; CRL-2159) tumor cell lines were maintained in vitro as monolayer in RPMI 1640 medium supplemented with 10% heat inactivated FBS, at 37° C. in an atmosphere of 5% C02 in air. The tumor cells were sub-cultured, not exceeding 4-5 passages, and cells growing in an exponential growth phase were harvested and counted for tumor inoculation. Each mouse was inoculated subcutaneously on the right flank with LS411N tumor cells (2×10⁶) in 0.1 mL of RPMI-1640 with Matrigel (1:1) for model development.

Depending on the target concentration, DHX9 compound was dissolved in in 0.96 mL Solutol while vortexing and sonicating for 1 hour to obtain a suspension. Then 2.4 mL of 1% MC (with 100 mg/mL PVP VA64) was added and vortexed for 5 min to obtain homogeneous suspension. The mixture was kept stirring at room temperature for 5 min, and then 1.44 mL of water was added while vortexing to make the final volume of 4.8 mL. The mixture was kept stirring at RT for 30 min and homogenized for 3 min. The resulting solution was used for in vivo studies.

Treatment was started when the mean tumor size reached approximately 100-150 mm³, at which time the mice were randomized into treatment groups. Animals were then treated with vehicle (20% Solutol/50% 1% MC (4000 cp) with 100 mg/mL PVP VA64/30% water) or indicated mg/kg of DHX9 compound (Compounds 1, 2, 3 and 4, as shown in Table 1) daily BID (12 hour schedule) by oral gavage at a final dosing volume of 10 mL/kg.

Tumor samples 2-3 mm³ in size were transferred into a clean tube and 300 μL of fresh lysis buffer containing 1% 2-

0.12mercaptoethanol was added. The samples were then homogenized using a TissueLyser at a frequency of 30/s for 5 min. The homogenate samples are then centrifuged at 1200 rpm for 15 min. The supernatant was transferred to a clean tube, and one volume of 70% ethanol to each volume of homogenate supernatant is added, and the samples are then vortexed.

RNA is then purified using the PureLink RNA Mini Kit according to the manufacturer's (Invitrogen) protocol, and cDNA is reverse transcribed using the High Capacity RNA-to-cDNA Kit according to the manufacturer's (Invitrogen) protocol. Using TaqMan Multiplex Mastermix and thermal cycler conditions for the QuantStudio Thermocycler, qPCR is carried out using TaqMan Gene Expression Assay with VIC Dye for Linear BRIP1 (Assay ID: Hs00908156_m1), TaqMan GAPDH primer limited assay with JUN dye/QSY probe, and the below custom TaqMan Gene Expression Assay with FAM Dye for circBRIP1:

•Forward Primer:
(SEQ ID NO: 19)
GTGAGCCAAGGAATTTTGTGTTT
•Reverse Primer:
(SEQ ID NO: 20)
GGTCTGAACTTTGCCATTAATATCTG
•Probe:
(SEQ ID NO: 21)
CCATCTTACAAGGCCTTT

Note: All target genes can be combined in one well per sample.

Data analysis was performed by using the QuantStudio Thermocycler software to determine threshold Ct values. ΔCt for circBRIP1 or linBRIP1 is calculated by normalizing the target gene Ct to the GAPDH housekeeping gene Ct for each sample. The inhibition activity is calculated by the following formula, whereas:

ΔCts: Mean of Ct(target gene)−Mean of Ct(GAPDH) for each sample

ΔCtc: Mean of Ct(target gene)−Mean of Ct(GAPDH) for DMSO control.

$$\% Vehicle = 100 \% \times \frac{{2}^{- Δ Cts}}{{2}^{- Δ Ctc}}$$

### Results

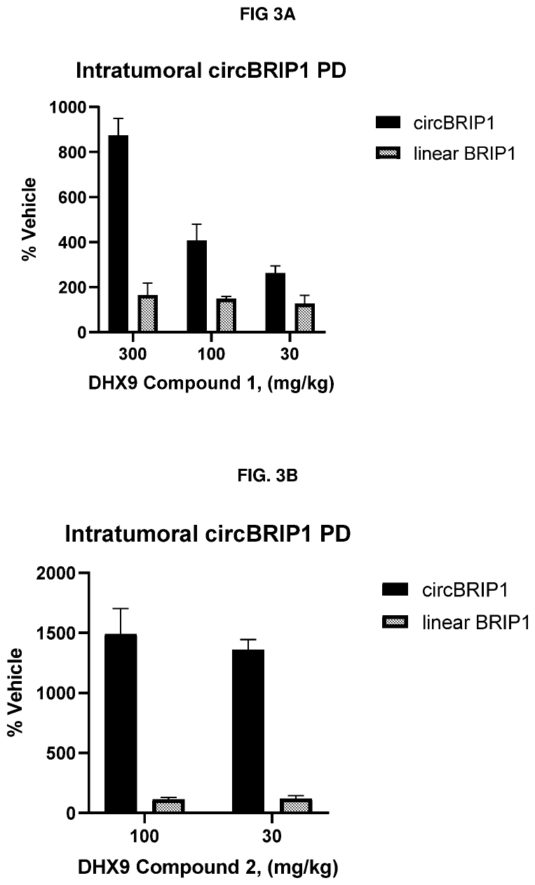

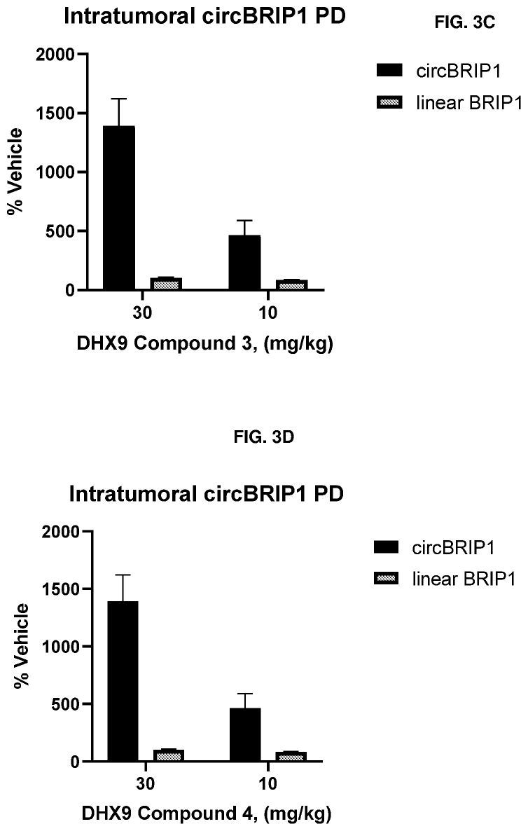

In tumors of mice treated with any of the 4 DHX9 inhibitor compounds, a dose dependent induction of circBRIP1 was observed and no change in linear BRIP1 was seen (see FIGS. 3A-3D).

### Example 4: Correlations of Intra-Tumoral circBRIP1 with Anti-Tumor Activity of DHX9 Inhibitors In Vivo

This study was conducted to evaluate pharmacodynamics (PD) and pharmacokinetics (PK) of intra-tumoral circular BRIP1 with efficacy of a DHX9 inhibitor (Compound 1, as shown in Table 1).

### Methods

In vivo Efficacy Study: Experiments were performed in female BALB/c nude mice (GenPharmatech Co.). Animals were allowed to acclimate for 7 days before the study. The general health of the animals were evaluated by a veterinarian, and complete health checks were performed prior to the study. General procedures for animal care and housing were in accordance with the standard, Commission on Life Sciences, National Research Council, Standard Operating Procedures (SOPs) of Pharmaron, Inc. The mice were kept in laminar flow rooms at constant temperature and humidity with 3-5 mice in each cage. Animals were housed in polycarbonate cage which is the size of 300×180×150 mm³ and in an environmentally monitored, well-ventilated room maintained at a temperature of (23±3° C.) and a relative humidity of 40% 70%. Fluorescent lighting provided illumination approximately 12 hours per day. Animals had free access to irradiation sterilized dry granule food during the entire study period except for time periods specified by the protocol, as well as sterile drinking water in a bottle was available ad libitum during the quarantine and study periods.

The LS411N (ATCC; CRL-2159) tumor cell lines were maintained in vitro as monolayer in RPMI 1640 medium supplemented with 10% heat inactivated FBS, at 37° C. in an atmosphere of 5% CO2 in air. The tumor cells were sub-cultured, not exceeding 4-5 passages, and cells growing in an exponential growth phase were harvested and counted for tumor inoculation. Each mouse was inoculated subcutaneously on the right flank with LS411N tumor cells (2×10⁶) in 0.1 mL of RPMI-1640 with Matrigel (1:1) for model development.

Depending on the target concentration, 14.4-144 mg DHX9 compound was dissolved in in 0.96 mL Solutol while vortexing and sonicating for 1 hour to obtain a suspension. Then 2.4 mL of 1% MC (with 100 mg/mL PVP VA64) was added and vortexed for 5 min to obtain homogeneous suspension. The mixture was kept stirring at room temperature for 5 min, and then 1.44 mL of water was added while vortexing to make the final volume of 4.8 mL. The mixture was kept stirring at RT for 30 min and homogenized for 3 min. The resulting solution was used for in vivo studies.

Treatment was started when the mean tumor size reached approximately 100-150 mm³, at which time the mice were randomized into treatment groups. Animals were then treated with vehicle (20% Solutol/50% 1% MC (4000 ep) with 100 mg/mL PVP VA64/30% water) or indicated mg/kg of DHX9 compound 1 daily BID (12 hour schedule) by oral gavage at a final dosing volume of 10 mL/kg.

The measurement of tumor size was conducted with a caliper and recorded twice per week. The tumor volume (TV) (mm3) was estimated using the formula: TV=a×b2/2, where “a” and “b” are long and short diameters of a tumor, respectively.

Intra-tumoral Pharmacokinetics Bioanalytical Assay: Tumors were collected at 1 h and 12 h post the last dose and cut into 3 pieces. One piece of the tumor was weighted and snap frozen for drug exposure analysis. On the assay day, 3 volumes of H2O were added to the tumor tissue (3 mL to each gram of tissue), and homogenate at 4 degrees Celsius. The homogenate (30 μL) was mixed with 15 μL blank solution, then extracted and protein precipitation with 200 μL of acetonitrile containing IS (internal standard, dexamethasone). After vortexed for 30 s, the samples were centrifuged at 4 degrees Celsius, 3900 rpm for 15 minutes. The supernatant was diluted 3 times with water and 2 μL of diluted supernatant was injected into the LC/MS/MS system for quantitative analysis (AB API 5500). The Q1 to Q3 mass transition (m/z, Da) were 393.11 to 373.10 for dexamethasone and 421.98 to 202.30 for test compound. HPLC separation was achieved on an Agilent poroshell EC-C18 4 μm (50×2.1 mm) column with gradient from 5% Acetonitrile in Water (0.1% Formic acid) to 95% Acetonitrile in Water (0.1% Formic acid) in 2.5 minutes. HPLC: Shimadzu system (DGU-20A5R, LC-30AD, SIL-30AC, Rack Changer II, CTO-30A, and CBM-20A).

The concentration in the homogenate was quantified using standards prepared in control tumor tissues with concentrations range 0.5, 1, 2, 5, 10, 50, 100, 500, 1000 ng/mL. The determined concentration in the tumor homogenate was then back calculated to that in tumor tissue by multiplying with a dilution factor of 4.

Intra-tumoral Human circBRIP1 Analysis: Tumor samples 2-3 mm³ in size were transferred into a clean tube and 300 μL of fresh lysis buffer containing 1% 2-mercaptoethanol was added. The samples were then homogenized using a TissueLyser at a frequency of 30/s for 5 min. The homogenate samples are then centrifuged at 1200 rpm for 15 min. The supernatant was transferred to a clean tube, and one volume of 70% ethanol to each volume of homogenate supernatant was added, and the samples were then vortexed.

RNA was then purified using the PureLink RNA Mini Kit according to the manufacturer's (Invitrogen) protocol, and cDNA is reverse transcribed using the High Capacity RNA-to-cDNA Kit according to the manufacturer's (Invitrogen) protocol. Using SYBR Green Master Mix (Invitrogen) and thermal cycler conditions for the QuantStudio Thermocycler, qPCR was carried out using the below custom primer sequences:

•BRIP1-Circular-Forward
(SEQ ID NO: 9)
(TCT GTG TGC CAG ACT GTG AG)
•BRIP1-Circular-Reverse
(SEQ ID NO: 10)
(ACA CCA AGT TCT GAC GAA AAG G)
•GAPDH-Forward
(SEQ ID NO: 17)
(GTCTCCTCTGACTTCAACAGCG)
•GAPDH-Reverse
(SEQ ID NO: 18)
(ACCACCCTGTTGCTGTAGCCAA)

Note: Forward and Reverse primers are combined per gene, and each sample has to be run on a separate plate for each gene.

Data analysis was performed by using the QuantStudio Thermocycler software to determine threshold Ct values. ΔCt for circBRIP1 or linBRIP1 is calculated by normalizing the target gene Ct to the GAPDH housekeeping gene Ct for each sample. The inhibition activity is calculated by the following formula, whereas:

ΔCts: Mean of Ct(target gene)−Mean of Ct(GAPDH) for each sample

ΔCtc: Mean of Ct(target gene)−Mean of Ct(GAPDH) for DMSO control.

$$\% Vehicle = 100 \% \times \frac{{2}^{- Δ Cts}}{{2}^{- Δ Ctc}}$$

### Results

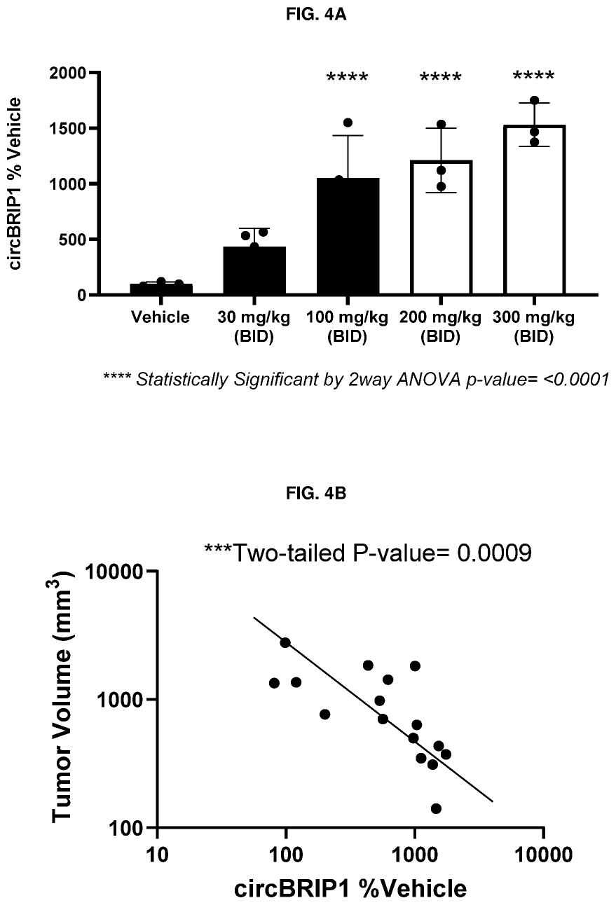

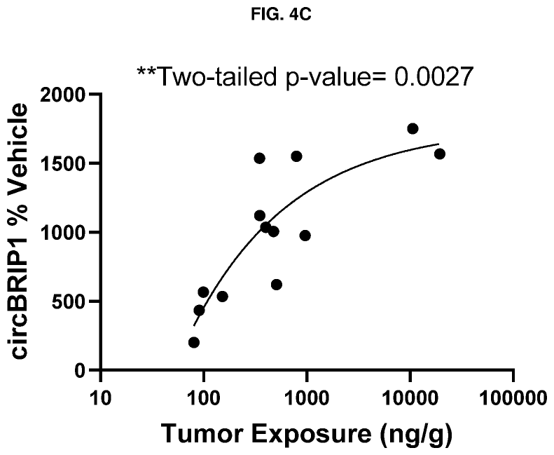

Compound 1 was treated at 30, 100, 200 and 300 mg/kg using BID oral application when the tumor volume was an average of 133 mm3 (n=8/group). After 21 days of treatment and 12 hours post last dose of compound tumor volume was recorded, tumors were then harvested and processed for circBRIP1 expression. Dose-dependent intra-tumoral circBRIP1 induction was observed, and was statistically significant compared to vehicle control based on two way ordinary ANOVA test, with indicated doses resulting in p-values <0.0001 (see FIG. 4A). Individual tumor volumes were plotted against each tumor's corresponding circBRIP1 pharmacoynamic reading in a correlation plot (see FIG. 4B). Individual tumor drug exposure of Compound 1 were plotted against each tumor's corresponding circBRIP1 pharmacodynamic reading and fit to four parameter non-linear regression (see FIG. 4C). These results show that both tumor volume and tumor drug exposure correlate to circBRIP1 induction.

### Example 5: Evaluation of circBRIP1 in Peripheral Blood Mononuclear Cells (PBMC) of Mice

This study was conducted to evaluate pharmacodynamics (PD) of the circular format of mouse BRIP1 mRNA in mouse PBMC cells by qPCR.

### Methods

Experiments were performed in female BALB/c nude mice (GenPharmatech Co.). Animals were allowed to acclimate for 7 days before the study. The general health of the animals were evaluated by a veterinarian, and complete health checks were performed prior to the study. General procedures for animal care and housing were in accordance with the standard, Commission on Life Sciences, National Research Council, Standard Operating Procedures (SOPs) of Pharmaron, Inc. The mice were kept in laminar flow rooms at constant temperature and humidity with 3-5 mice in each cage. Animals were housed in polycarbonate cage which is the size of 300×180×150 mm³ and in an environmentally monitored, well-ventilated room maintained at a temperature of (23±3° C.) and a relative humidity of 40% 70%. Fluorescent lighting provided illumination approximately 12 hours per day. Animals had free access to irradiation sterilized dry granule food during the entire study period except for time periods specified by the protocol, as well as sterile drinking water in a bottle was available ad libitum during the quarantine and study periods.

The LS411N (ATCC; CRL-2159) tumor cell lines were maintained in vitro as monolayer in RPMI 1640 medium supplemented with 10% heat inactivated FBS, at 37° C. in an atmosphere of 5% CO2 in air. The tumor cells were sub-cultured, not exceeding 4-5 passages, and cells growing in an exponential growth phase were harvested and counted for tumor inoculation. Each mouse was inoculated subcutaneously on the right flank with LS411N tumor cells (2×106) in 0.1 mL of RPMI-1640 with Matrigel (1:1) for model development.

Depending on the target concentration, DHX9 compound (Compound 2, 3, or 4, as shown in Table 1) was dissolved in in 0.96 mL Solutol while vortexing and sonicating for 1 hour to obtain a suspension. Then 2.4 mL of 1% MC (with 100 mg/mL PVP VA64) was added and vortexed for 5 min to obtain homogeneous suspension. The mixture was kept stirring at room temperature for 5 min, and then 1.44 mL of water was added while vortexing to make the final volume of 4.8 mL. The mixture was kept stirring at RT for 30 min and homogenized for 3 min. The resulting solution was used for in vivo studies.

Treatment was started when the mean tumor size reached approximately 100-150 mm³, at which time the mice were randomized into treatment groups. Animals were then treated with vehicle (20% Solutol/50% 1% MC (4000 cp) with 100 mg/mL PVP VA64/30% water) or indicated mg/kg of DHX9 compound daily BID (12 hour schedule) by oral gavage at a final dosing volume of 10 mL/kg.

Whole blood was collected into tubes coated with EDTA-K2. 1×PBS (volume ratio=1:1) to blood sample was added and mixed gently. The blood sample was transferred into a new tube containing Ficoll-Paque™ PREMIUM 1.084 (volume ratio=1:5), and centrifuged at 400 g for 20 min at room temperature. The layer of lymphocytes was transferred into a new 1.5 mL tube containing 0.5 mL of 1×PBS, and centrifuged at 1500 rpm for 5 min. The samples were then washed twice with 1.0 ml of 1×PBS, and red blood cells were lysed with 1× red blood cell lysing buffer. Resulting isolated PBMC were then suspended in 1×PBS for further analysis.

PBMC samples were transferred into a clean tube and 300 μL of fresh lysis buffer containing 1% 2-mercaptoethanol. The samples were then homogenized using a TissueLyser at a frequency of 30/s for 5 min. The homogenate samples were then centrifuged at 1200 rpm for 15 min. The supernatant was transferred to a clean tube, and one volume of 70% ethanol to each volume of homogenate supernatant was added, and the samples were then vortexed. RNA was purified using the PureLink RNA Mini Kit according to the manufacturer's (Invitrogen) protocol, and cDNA was reverse transcribed using the High Capacity RNA-to-cDNA Kit according to the manufacturer's (Invitrogen) protocol. Using TaqMan Multiplex Mastermix (Thermo Fisher) and thermal cycler conditions for the QuantStudio Thermocycler, qPCR was carried out using TaqMan Gene Expression Assay with FAM Dye for Mouse linear BRIP1 (Assay ID: Mm01297848_m1), TaqMan mouse GAPDH primer limited assay with VIC dye (Assay ID: Mm99999915_g1), and the below custom TaqMan Gene Expression Assay with FAM Dye for mouse circBRIP1:

•Forward Primer:
(SEQ ID NO: 22)
TCAAAATAGAAGCCAGTGGATGAG
•Reverse Primer:
(SEQ ID NO: 23)
AATGGCATGCACAAGAACATG
•Probe:
(SEQ ID NO: 24)
CCTGAGGCACCACCT

Note: each sample needed to be run in 2 different plates, one with mouse linear BRIP1 and GAPDH, and the other plate with mouse circBRIP1 and GAPDH.

Data analysis was performed by using the QuantStudio Thermocycler software to determine threshold Ct values. ΔCt for circBRIP1 or linBRIP1 was calculated by normalizing the target gene Ct to the GAPDH housekeeping gene Ct for each sample. The inhibition activity was calculated by the following formula, whereas:

ΔCts: Mean of Ct(target gene)−Mean of Ct(GAPDH) for each sample

ΔCtc: Mean of Ct(target gene)−Mean of Ct(GAPDH) for DMSO control.

$$\% Vehicle = 100 \% \times \frac{{2}^{- Δ Cts}}{{2}^{- Δ Ctc}}$$

### Results

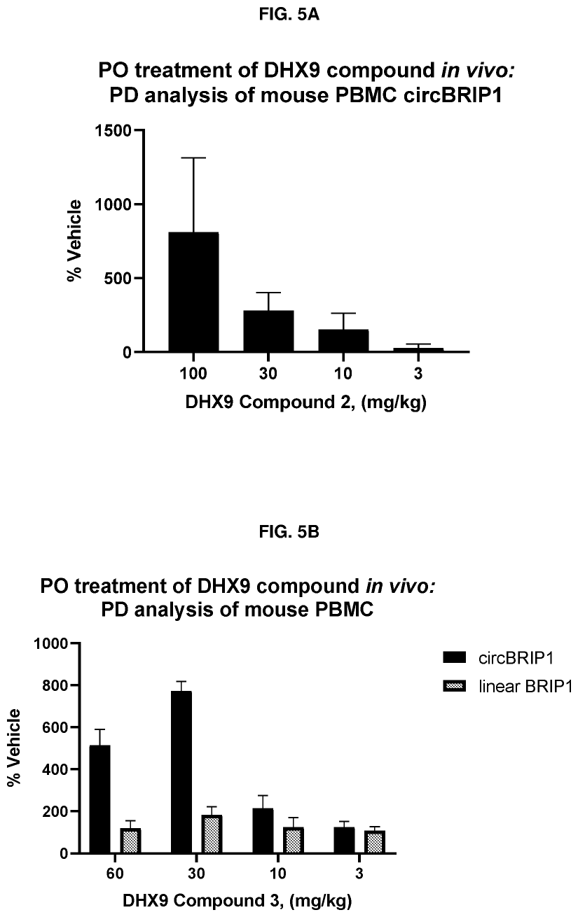

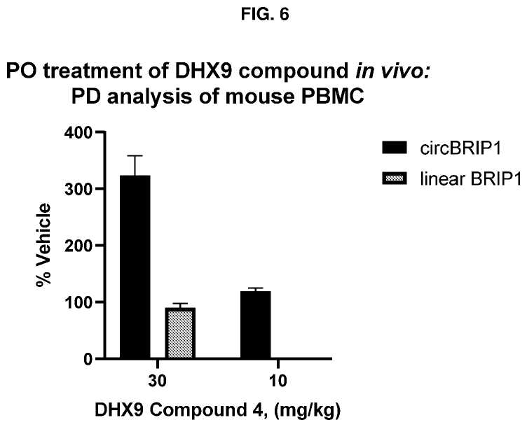

At 1 hr (FIGS. 5A-B) and 24 hours (FIG. 6) after the last dose of DHX9 inhibitor, dose dependent induction of circBRIP1 was observed, and no induction in linear BRIP1 above baseline was seen.

### Example 6: Comparison of circBRIP1 Induction by DHX9 Inhibitor and Sensitivity to DHX9 Inhibitor in Cancer Cell Lines

This study was conducted to determine inhibition of DHX9 in a range of cancer cell lines and whether circBRIP1 correlates with sensitivity to DHX9 inhibition.

### Methods

Cells were plated at pre-determined cell densities in 384-well cell culture plates and incubated overnight in 37° C. at 5% CO₂. A number of colorectal cancer (CRC) cell lines, including MSI-H CRC cells (sensitive to DHX9 inhibitor) and MSS CRC cells (insensitive to DHX9 inhibitor) were treated with escalating doses of from 0.001 to 10 μM Compound 1, including HCT116 (CRC MSI; Sensitive), HT-29 (CRC MSS; Insensitive), DLD-1 (CRC MSI; Sensitive), HCT-15 (CRC MSI; Sensitive), LS411N (CRC MSI; Sensitive), NCI-H747 (CRC MSS; Insensitive), LS174T (CRC MSI; Sensitive), LOVO (CRC MSI; Sensitive), SW48 (CRC MSI; Insensitive), and SNU-407 (CRC MSI; Insensitive) Reference and test compounds were prepared in DMSO stock solution and serially diluted 3-fold for 10 points. Compound 1 (0.1% final DMSO concentration) was transferred to the cell plate in designated wells, and incubated 37° C. at 5% CO₂ for 72 hours. Compound 1 was prepared to a 10 mM stock solution, and 10 tested doses were created by titration by diluting the compound 1 fold each step. RNA was extracted from the cells using the TaqMan Gene Expression Cells-to-CT™ kit according to manufacturer's (Invitrogen) protocol, and cDNA was reverse transcribed using the High Capacity RNA-to-cDNA Kit according to the manufacturer's (Invitrogen) protocol. Using TaqMan Multiplex Mastermix and thermal cycle conditions for the Quant Studio Thermocycler, qPCR was carried out as described above to quantify the level of circBRIP1.

Data analysis was performed by using the QuantStudio Thermocycler software to determine threshold Ct values. ΔCt for circBRIP1 is calculated by normalizing the target gene Ct to the GAPDH housekeeping gene Ct for each sample.

The inhibition activity (% Vehicle) is calculated by the following formulas, whereas:

ΔCts: Mean of Ct(target gene)−Mean of Ct(GAPDH) for each sample

ΔCtc: Mean of Ct(target gene)−Mean of Ct(GAPDH) for DMSO control

$$mRNA level = 2 - Δ Ctc$$

$$\% Vehicle = 100 \times [ mRNA ( compound treated ) / mRNA ( DMSO ) ]$$

An EC50 was then calculated using a 4-parameter logistic nonlinear regression model in Scigilian Analyze or GraphPad Prism, floating both the top and bottom parameters.

$$EC 50 Fit formula = Y = A + ( B - A ) / ( 1 + 10 \hat{} ( ( Log C - X ) * D ) )$$

$$whereas A : Bottom ; B : Top ; C : EC 50 ; D : Slope$$

### Results

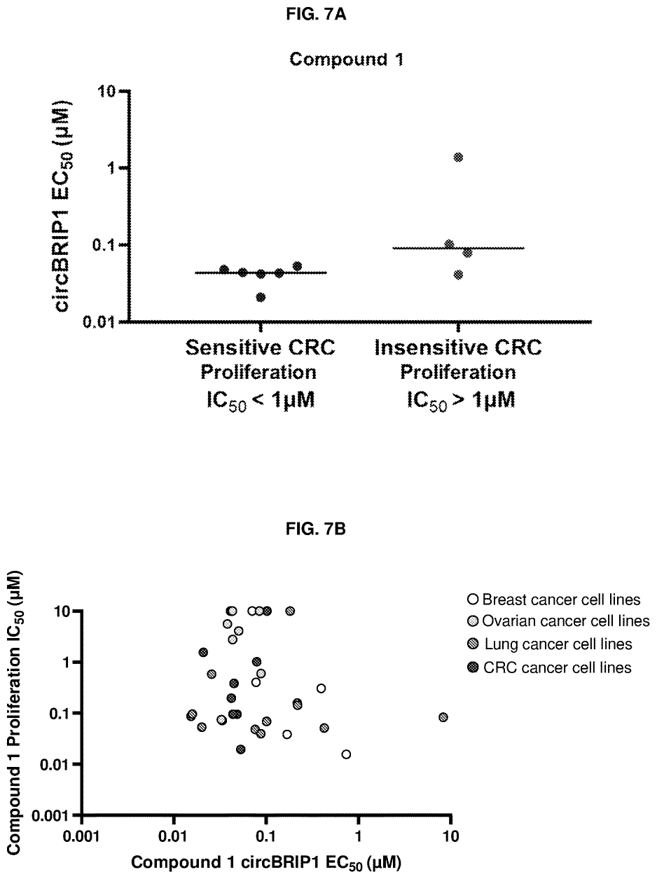

Induction of circBRIP1 was observed in both the DHX9-dependent and independent CRC cell lines with similar potency (see FIG. 7A). Thus, induction of circBRIP1 can serve as a clear and robust DHX9-specific target engagement biomarker across DHX9 sensitive and insensitive cells.

A similar experiment was carried out using assays as described above, testing levels of circBRIP1 and inhibition of proliferation for breast, ovarian, lung, and colorectal cancer (CRC) cell lines after treatment with DHX9 inhibitor (Compound 1). Consistent with the observations made in CRC cell lines, circBRIP1 levels were also upregulated in breast, ovarian, and lung cell lines, and the levels of circBRIP1 did not correlate with anti-proliferation effects of the DHX9 inhibitor.

In summary, induction of circBRIP1 can be used as a biomarker for DHX9 inhibition across DHX9 sensitive and insensitive cells.

### Example 7: Evaluation of circBRIP1 in Frozen Human PBMCs

As proof of concept, circBRIP1 levels were examined in peripheral blood mononuclear cells (PMBCs) using frozen PBMCs from donors after treatment with a DHX9 inhibitor.

Frozen PBMCs were thawed in a 37° C. water bath, washed, and reconstituted in media. The same number of cells were added to each well of 12-well plates and let rest for 2 hours before being subjected to testing conditions. Compound 1 was dissolved in DMSO at 10 mM as a stock solution, and diluted to a desired concentration. Frozen PBMCs from three donors or LS411N cells (positive control) were treated with 10 μM of Compound 1 or control vehicle for 24 hours. The circBRIP1 level was determined using methods essentially as described above.

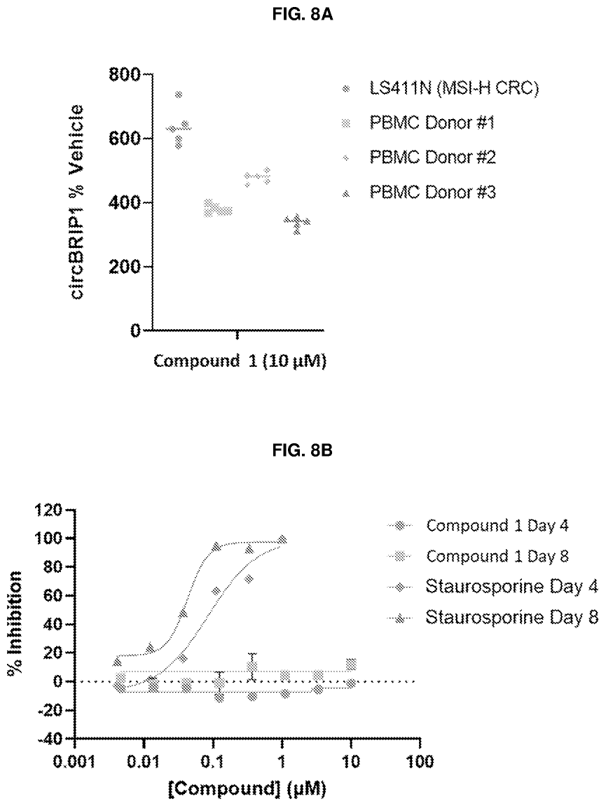

As shown in FIG. 8A, treatment with Compound 1 significantly elevated the circBRIP1 level in PBMCs as compared to vehicle-treated cells.

Next, the proliferation of PMBCs was examined following treatment with DHX9 inhibitor using the following assay.

Human PBMCs were thawed from a frozen cryotube in a 37° C. water bath and transferred to a flask with medium X-Vivo 15 supplemented with 5% Fetal Bovine Serum (FBS), 1% PenStrep, and 1% Glutamax. Flasks were incubated at 37 C for 30 minutes. PBMCs were harvested from flask and counted. PBMCs were diluted in medium and added to 96 well plates at 100,000 PBMCs per well. A stimulus mixture containing IL-15 at 1 ng/well, IL-21 at 5 ng/well, and CD40L at 5 ng/well was added to each well. Test compounds were prepared in DMSO stock solution and serially diluted. Compound (0.1% final DMSO concentration) was added to each well. DMSO was used as a negative control (high control, HC) and staurosporine was used a positive control (low control, LC). Plates were incubated at 37 C for 0, 2, 4, 6, 8, 10 days. Every other day, medium was replenished with stimulus and diluted compounds.

At each CTG time point, plate was removed from incubator and Cell-Titer Glo 2.0 reagent was added to each well according to manufacturer's (Promega) protocol and detected on an Envision. The inhibition activity was calculated following the formula below:

% Inhibition=100×(ReadoutHC−ReadoutSample)/(ReadoutHC−ReadoutLC)

PBMCs were exposed to Compound 1 at concentrations ranging from 0.01 μM to 10 μM for 10 days. PMBCs were treated with staustoporine as positive control for inhibition.

As shown in FIG. 8B, staustoporine inhibited proliferation of PBMCs. Surprisingly, Compound 1 did not inhibit proliferation of PBMCs through the duration tested.

In summary, treatment of PBMCs elevated circBRIP1 level in the cells, but did not impact their proliferation.

### Example 8: Evaluation of circBRIP1 in Human PBMCs

This study was conducted to evaluate the circular format of BRIP1 mRNA in fresh human PBMC cells by qPCR.

### Methods

Human whole blood was obtained from Research Blood Components. Blood of each donor was aliquoted into vacutainer tubes containing Acid Citrate Dextrose (ACD).

Test compounds were prepared in DMSO stock solution and serially diluted. Compound (0.1% final DMSO concentration) was added to vacutainer tubes containing human whole blood. Tubes were placed in tube rotator and incubated at 37 C for 24 hours.

PBMCs were isolated from human whole blood after compound treatment by density gradient centrifugation using Ficoll-Paque Plus. RNA was extracted from PBMCs using RNeasy Plus Kits according to the manufacturer's (Qiagen) protocol. cDNA was reverse transcribed using RT² First Strand Kit according to the manufacturer's (Qiagen) protocol. Using TaqMan Multiplex Mastermix and thermal cycler conditions for the QuantStudio Thermocycler, qPCR was carried out using TaqMan Gene Expression Assay with VIC Dye for Linear BRIP1 (Assay ID: Hs00908156_m1), TaqMan GAPDH primer limited assay with JUN dye/QSY probe, and the below custom TaqMan Gene Expression Assay with FAM Dye for circBRIP1:

•Forward Primer:
(SEQ ID NO: 19)
GTGAGCCAAGGAATTTTGTGTTT
•Reverse Primer:
(SEQ ID NO: 20)
GGTCTGAACTTTGCCATTAATATCTG
•Probe:
(SEQ ID NO: 21)
CCATCTTACAAGGCCTTT

Note: All target genes can be combined in one well per sample.

Data analysis was performed by using the QuantStudio Thermocycler software to determine threshold Ct values. ΔCt for circBRIP1 or linBRIP1 was calculated by normalizing the target gene Ct to the GAPDH housekeeping gene Ct for each sample. The inhibition activity was calculated by the following formula, whereas:

ΔCts: Mean of Ct(target gene)−Mean of Ct(GAPDH) for each sample

ΔCtc: Mean of Ct(target gene)−Mean of Ct(GAPDH) for DMSO control.

$$\% Vehicle = 100 \% \times \frac{{2}^{Δ Cts}}{{2}^{Δ Ctc}}$$

### Results

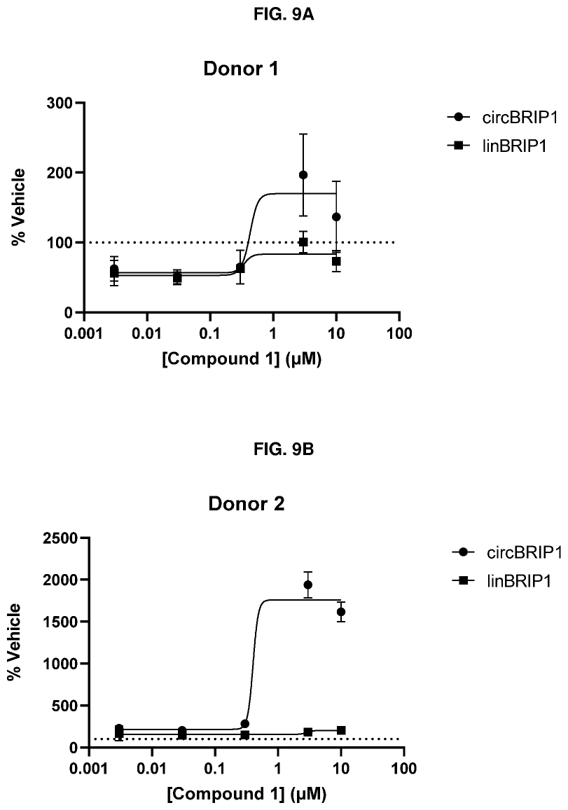

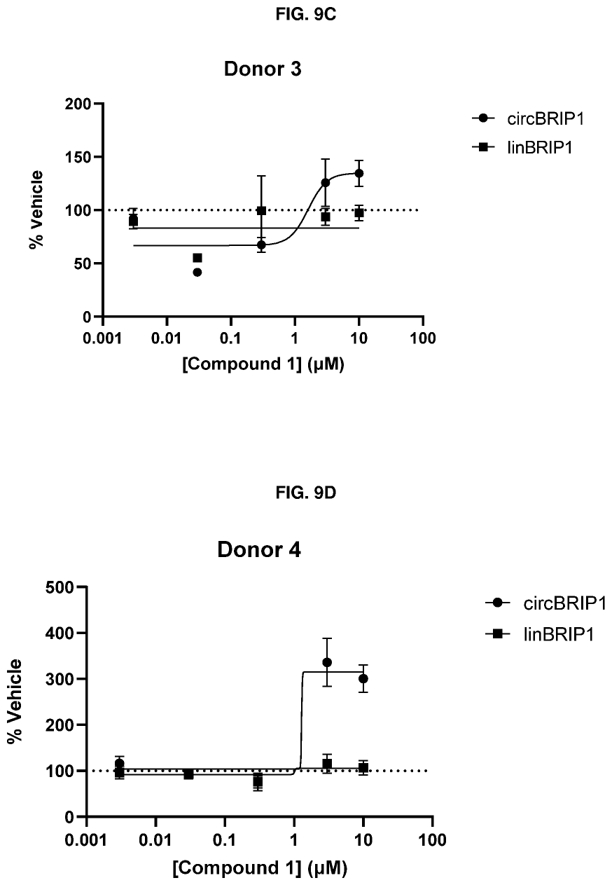

In all donor's blood, treatment with DHX9 inhibitor compound 1 at varying concentrations induced the presence of human circBRIP1 in a dose-responsive manner. Linear BRIP1 was not significantly changed (see FIGS. 9A-9D).

This result indicates that circBRIP1 in PBMCs may be used to monitor DHX9 target engagement and inhibition in vivo.

### Example 9: Evaluation of circBRIP1 in Human Whole Blood

This study is conducted to evaluate the circular format of BRIP1 mRNA in fresh human whole blood by qPCR.

### Methods

Human whole blood IS obtained from Research Blood Components. Blood of each donor IS aliquoted into vacutainer tubes containing Acid Citrate Dextrose (ACD).

Test compounds are prepared in DMSO stock solution and serially diluted. Compound (0.1% final DMSO concentration) is added to vacutainer tubes containing human whole blood. Tubes are placed in tube rotator and incubated at 37 C for 24 hours.

Total cells are isolated from human whole blood after compound treatment by density gradient centrifugation using Ficoll-Paque Plus. RNA is extracted from cells using RNeasy Plus Kits according to the manufacturer's (Qiagen) protocol. cDNA is reverse transcribed using RT² First Strand Kit according to the manufacturer's (Qiagen) protocol. Using TaqMan Multiplex Mastermix and thermal cycler conditions for the QuantStudio Thermocycler, qPCR is carried out using TaqMan Gene Expression Assay with VIC Dye for Linear BRIP1 (Assay ID: Hs00908156_m1), TaqMan GAPDH primer limited assay with JUN dye/QSY probe, and the below custom TaqMan Gene Expression Assay with FAM Dye for circBRIP1:

•Forward Primer:
(SEQ ID NO: 19)
GTGAGCCAAGGAATTTTGTGTTT
•Reverse Primer:
(SEQ ID NO: 20)
GGTCTGAACTTTGCCATTAATATCTG
•Probe:
(SEQ ID NO: 21)
CCATCTTACAAGGCCTTT

Note: All target genes can be combined in one well per sample.

Data analysis is performed by using the QuantStudio Thermocycler software to determine threshold Ct values. ΔCt for circBRIP1 or linBRIP1 is calculated by normalizing the target gene Ct to the GAPDH housekeeping gene Ct for each sample. The inhibition activity is calculated by the following formula, whereas:

ΔCts: Mean of Ct(target gene)−Mean of Ct(GAPDH) for each sample

ΔCtc: Mean of Ct(target gene)−Mean of Ct(GAPDH) for DMSO control.

$$\% Vehicle = 100 \% \times \frac{{2}^{Δ Cts}}{{2}^{Δ Ctc}}$$

### Results

It is expected that treatment with DHX9 inhibitor compound 1 at varying concentrations will induce the presence of human circBRIP1 in a dose-responsive manner, with linear BRIP1 not significantly changed, suggesting that circBRIP1 in whole blood may be used to monitor DHX9 target engagement and inhibition in vivo.

### Example 10: Identification of Additional Alu-circRNAs Mediated by DHX9

This study was conducted to identify additional Alu-mediated circRNAs that can be markers for evaluating DHX9 inhibition.

### Methods

Screening for circRNA upregulated after knockdown of DHX9: HCT116 CRC-MSI cells were treated with DHX9 siRNA for a total of 3 days. Cells were harvested, and lysed with 1 ml of the TRIzol Reagent per 5-10×10⁶ cells. Total RNA from each sample was quantified using the NanoDrop ND-1000. The sample preparation and microarray hybridization were performed based on the Arraystar Human Circular RNA Array's standard protocols. Briefly, total RNAs were digested with Rnase R (Epicentre, Inc.) to remove linear RNAs and enrich circular RNAs. Then, the enriched circular RNAs were amplified and transcribed into fluorescent cRNA utilizing a random priming method (Arraystar Super RNA Labeling Kit; Arraystar). The labeled cRNAs were hybridized onto the Arraystar Human circRNA Array V2 (8×15K, Arraystar). After having washed the slides, the arrays were scanned by the Agilent Scanner G2505C.

Agilent Feature Extraction software (version 11.0.1.1) was used to analyze acquired array images. Quantile normalization and subsequent data processing were performed using the R software limma package. Differentially expressed circRNAs with statistical significance between two groups were identified through Volcano Plot filtering. Differentially expressed circRNAs between two samples (DHX9 siRNA-treated and scrambled control siRNA treated) were identified through Fold Change filtering. Hierarchical Clustering was performed to show the distinguishable circRNAs expression pattern among samples.

Identifying Alu-mediated circRNA among circRNAs upregulated by knockdown of DHX9: Up-regulated circRNA that were found to be differentially regulated by DHX9 knockdown were categorized as Alt mediated by methods as described by Stagsted et al. 2019 (Noncoding AUG circRNAs constitute an abundant and conserved subclass of circles, Life Science Alliance, 2019, 2(3):1-16), the entire contents of which are incorporated herein by reference). Stagsted et al. states that Alu-mediated circRNA biogenesis occurs by aberrant backsplicing of RNA transcripts as stimulated by bringing Inverted Alu Elements (IAE) into close proximity with each other. Through a different mechanism, circRNA can also occur when long flanking introns backsplice on each other, which is termed “AUG circRNA” and relies on IAE-independent (i.e., Alu-independent) mode of biogenesis, whereas “other circRNA” relies on IAE-dependent biogenesis. Stagsted et al. shows through High-Throughput Sequencing of RNA isolated by CrossLinking ImmunoPrecipitation (HITS-CLIP) that DHX9 occupancy shows a clear selection for binding at proximal flanking regions of Alu-dependent circRNAs, as well as a clear preference for non-AUG over AUG circRNA.

CircRNAs up-regulated by DHX9 knockdown identified by the Arraystar Human Circular RNA Array was compared to a list of circRNAs sensitive to DHX9 expression provided in Supplemental Table 4 of Stagsted et al. 2019. CircRNAs annotated as “AUG circRNA” by Statsted et al. were considered to not be sensitive to DHX9 activity, and thus not dependent on IAE for biogenesis. CircRNAs annotated as “Ambiguous circRNA” were also irrelevant for this analysis, as they are divergent across multiple host genes

### Results

The analysis as described above yielded the circRNAs listed in Table 2 as Alu-mediated circRNAs useful in evaluating DHX9 inhibition in the methods and assays of the present disclosure.

Larger panels of circRNA other than those included in the Arraystar Human Circular RNA Array are tested using this same or similar approach to identify additional Alu-mediated circRNAs useful for the methods and assays provided herein

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of measuring activity of a DHX9 inhibitor in a cell, comprising:
contacting a cell with the DHX9 inhibitor; and
measuring the level of an Alu-mediated circular RNA (Alu-circRNA) in the cell.

2. The method of claim 1, wherein the cell is a cancer cell.

3. The method of claim 1, wherein the cell is a non-cancerous cell.

4. The method of claim 1, wherein the cell is a peripheral blood mononuclear cell (PBMC).

5. The method of any one of claims 1-3, wherein the contacting is for a sufficient time for the Alu-circRNA to be produced.

6. The method of claim 5, wherein the level of Alu-circRNA is correlated to inhibition of a DHX9 activity by the DHX9 inhibitor in the cell.

7. A method of measuring activity of a DHX9 inhibitor in a subject, comprising:
administering a DHX9 inhibitor to a subject; and
measuring the level of an Alu-mediated circular RNA (Alu-circRNA) in a biological sample from the subject.

8. A method of measuring activity of a DHX9 inhibitor in a subject, comprising:
measuring the level of Alit-mediated circular RNA (Alu-circRNA) in a biological sample from a subject, wherein the subject is being treated with a DHX9 inhibitor.

9. The method of claim 7 or 8, wherein the level of Alu-circRNA is correlated to inhibition of DHX9 activity by the DHX9 inhibitor in the subject.

10. The method of claim 9, wherein the level of Alu-circRNA is correlated to inhibition of DHX9 ATPase activity or resolvase activity.

11. The method of claim 7 or 8, wherein the level of Alu-circRNA is correlated to inhibition of proliferation of MSI cancer cells in the subject.

12. The method of any one of claims 7-11, wherein the biological sample is selected from the group consisting of blood, interstitial fluid, lymph fluid, organ tissue, biopsy tissue, and skin tissue.

13. The method of claim 12 wherein the biological sample is blood.

14. The method of claim 13 wherein the method further comprises isolating cells from the blood sample, and measuring the level of the Alu-circRNA in the isolated cells.

15. The method of any one of claims 7-11, wherein the biological sample comprises peripheral blood mononuclear cells (PBMCs).

16. The method of claim 15, wherein the method further comprises isolating PBMCs from a blood sample, and measuring the level of the Alu-circRNA in the PBMCs.

17. The method of claim 15, wherein the level of Alu-circRNA does not correlate with inhibition of proliferation of PBMCs of the subject.

18. The method of claim any one of claims 1-17, wherein the Alu-circRNA is selected from the group consisting of cirRNA of BRIP1, AKR1A1, BMPR2, CALCOCO2, CLNSIA, DKC1, FAM124A, FBN1, FCHO2, GLS, GON4L, GPR125, NEIL3, PIK3C3, PNN, POMT1, RBM23, SKIL, SLK, SMA, SMG1, SMYD4, TAF2, TFAP4, TMEM41B, USP25, VPS13D, WDR59, and XPR1.

19. The method of any one of claims 1-18, wherein the level of the Alu-circRNA is measured by using one or more techniques selected from northernblot, reverse transcription-quantitative polymerase chain reaction (RT-qPCR), microarray, droplet digital PCR, next-generation sequence (NGS) RNA sequencing, RNA in situ hybridization (RNA-ISH), in situ hybridization-immunohistochemistry (ISH-IHC), isothermal exponential amplification, and rolling cycle amplification.

20. The method of any one of claims 7-19, further comprising obtaining the biological sample from the subject.

21. The method of any one of claims 7-20, wherein the biological sample is obtained from the subject between 0.5-7 days following administration of the DHX9 inhibitor.

22. The method of any one of claims 7-21, wherein the method comprises measuring the level of the Alu-circRNA in biological samples obtained from the subject at more than one time point.

23. The method of any one of claims 7-22, wherein the subject has a cancer.

24. The method of claim 23, wherein the cancer is a microsatellite instability (MSI) cancer.

25. The method of claim 23 or 24, wherein the cancer is selected from the group consisting of colorectal cancer, endometrial cancer, ovarian cancer, gastric cancer, hematopoietic cancer, breast cancer, brain cancer, skin cancer, lung cancer, blood cancer, prostate cancer, head and neck cancer, pancreatic cancer, bladder cancer, bone cancer, soft tissue cancer, kidney cancer, liver cancer, and Ewing's sarcoma.

26. The method of any one of claims 7-25, further comprising making a determination on an appropriate therapeutic dose of the DHX9 inhibitor based on a set of data including the level of Alu-circRNA measured.

27. The method of any one of claims 1-26, wherein the DHX9 inhibitor is selected from a small molecule, an antisense oligonucleotide, small interfering RNA (siRNA), a small hairpin RNA (shRNA), a microRNA (miRNA), or a piwiRNA (piRNA).

28. The method of any one of claims 1-26, wherein the DHX9 inhibitor is a small molecule.

29. The method of any one of claims 1-26, wherein the DHX9 inhibitor is selected from the DHX9 inhibitors in Table 1.

30. The method of any one of claims 7-29, wherein the subject is a human.

31. A method of monitoring treatment of a subject with a DHX9 inhibitor, comprising:
administering a DHX9 inhibitor to the subject; and
obtaining information as to the level of Alu-mediated circular RNA (Alu-circRNA) in a biological sample obtained from the subject after administration of the DHX9 inhibitor.

32. The method of claim 31, wherein the level of Alu-circRNA is correlated to inhibition of DHX9 activity by the DHX9 inhibitor in the subject.

33. The method of claim 32, wherein the level of Alu-circRNA is correlated to inhibition of DHX9 ATPase activity or resolvase activity.

34. The method of claim 31, wherein the level of Alu-circRNA is correlated to inhibition of proliferation of MSI cancer cells in the subject.

35. The method of any one of claims 31-34, wherein the biological sample is selected from the group consisting of blood, interstitial fluid, lymph fluid, organ tissue, biopsy tissue, and skin tissue.

36. The method of claim 35, wherein the biological sample is blood.

37. The method of claim 36, wherein the method further comprises isolating cells from the whole blood sample, and measuring the level of the Alu-circRNA in the isolated cells.

38. The method of any one of claims 31-34, wherein the biological sample comprises peripheral blood mononuclear cells (PBMCs).

39. The method of claim 38, wherein the method further comprises isolating PBMCs from a blood sample, and measuring the level of the Alu-circRNA in the PBMCs.

40. The method of claim 38 or 39, wherein the level of Alu-circRNA does not correlate with inhibition of proliferation of PBMCs cells of the subject.

41. The method of claim any one of claims 31-40, wherein the Alu-circRNA is selected from the group consisting of cirRNA of BRIP1, AKR1A1, BMPR2, CALCOCO2, CLNSIA, DKC1, FAM124A, FBN1, FCHO2, GLS, GON4L, GPR125, NEIL3, PIK3C3, PNN, POMT1, RBM23, SKIL, SLK, SMA, SMG1, SMYD4, TAF2, TFAP4, TMEM41B, USP25, VPS13D, WDR59, and XPR1.

42. The method of any one of claims 31-41, wherein the level of the Alu-circRNA is measured by using one or more techniques selected from northernblot, reverse transcription-quantitative polymerase chain reaction (RT-qPCR), microarray, droplet digital PCR, next-generation sequence (NGS) RNA sequencing, RNA in situ hybridization (RNA-ISH), in situ hybridization-immunohistochemistry (ISH-IHC), isothermal exponential amplification, and rolling cycle amplification.

43. The method of any one of claims 31-42, further comprising obtaining the biological sample from the subject.

44. The method of any one of claims 31-43, wherein the biological sample is obtained from the subject between 0.5-7 days following administration of the DHX9 inhibitor.

45. The method of any one of claims 31-44, wherein the method comprises measuring the level of the Alu-circRNA in biological samples obtained from the subject at more than one time point.

46. The method of any one of claims 31-45, wherein the subject has a cancer.

47. The method of claim 46, wherein the cancer is a microsatellite instability (MSI) cancer.

48. The method of claim 46 or 47, wherein the cancer is selected from the group consisting of colorectal cancer, endometrial cancer, ovarian cancer, gastric cancer, hematopoietic cancer, breast cancer, brain cancer, skin cancer, lung cancer, blood cancer, prostate cancer, head and neck cancer, pancreatic cancer, bladder cancer, bone cancer, soft tissue cancer, kidney cancer, liver cancer, and Ewing's sarcoma.

49. The method of any one of claims 31-48, further comprising making a determination on an appropriate therapeutic dose of the DHX9 inhibitor based on a set of data including the level of Alu-circRNA measured.

50. The method of any one of claims 31-49, wherein the DHX9 inhibitor is selected from a small molecule, an antisense oligonucleotide, small interfering RNA (siRNA), a small hairpin RNA (shRNA), a microRNA (miRNA), or a piwiRNA (piRNA).

51. The method of any one of claims 31-49, wherein the DHX9 inhibitor is a small molecule.

52. The method of any one of claims 31-49, wherein the DHX9 inhibitor is selected from the inhibitors in Table 1.

53. The method of any one of claims 31-49, wherein the subject is a human.


SEQUENCE LISTING (56 sequences)

SEQ ID NO: 1 - DNA, 20 bp, synthetic construct
  source 1..20: mol_type = other DNA
gaagactctggctgacctcc

SEQ ID NO: 2 - DNA, 20 bp, synthetic construct
  source 1..20: mol_type = other DNA
agcctacgctaagggcatac

SEQ ID NO: 3 - DNA, 20 bp, synthetic construct
  source 1..20: mol_type = other DNA
aggtccagcggaaagtgatc

SEQ ID NO: 4 - DNA, 20 bp, synthetic construct
  source 1..20: mol_type = other DNA
tgttcagggcatttagctgc

SEQ ID NO: 5 - DNA, 20 bp, synthetic construct
  source 1..20: mol_type = other DNA
ggatgagagttacctgcggc

SEQ ID NO: 6 - DNA, 20 bp, synthetic construct
  source 1..20: mol_type = other DNA
aatgtaggtgccagcctcac

SEQ ID NO: 7 - DNA, 20 bp, synthetic construct
  source 1..20: mol_type = other DNA
atgaggacggcattgaggtc

SEQ ID NO: 8 - DNA, 20 bp, synthetic construct
  source 1..20: mol_type = other DNA
gtcgcaggtagagatgaccg

SEQ ID NO: 9 - DNA, 20 bp, synthetic construct
  source 1..20: mol_type = other DNA
tctgtgtgccagactgtgag

SEQ ID NO: 10 - DNA, 22 bp, synthetic construct
  source 1..22: mol_type = other DNA
acaccaagttctgacgaaaagg

SEQ ID NO: 11 - DNA, 20 bp, synthetic construct
  source 1..20: mol_type = other DNA
gtccatcctgaggtagtcgg

SEQ ID NO: 12 - DNA, 20 bp, synthetic construct
  source 1..20: mol_type = other DNA
ttccccaggctgacaagttc

SEQ ID NO: 13 - DNA, 22 bp, synthetic construct
  source 1..22: mol_type = other DNA
tgaagaagatgaagttcggtat

SEQ ID NO: 14 - DNA, 20 bp, synthetic construct
  source 1..20: mol_type = other DNA
gtgccagatttctgagtttt

SEQ ID NO: 15 - DNA, 18 bp, synthetic construct
  source 1..18: mol_type = other DNA
gacagactctacgccatg

SEQ ID NO: 16 - DNA, 18 bp, synthetic construct
  source 1..18: mol_type = other DNA
gtctcccagcaagtgttc

SEQ ID NO: 17 - DNA, 22 bp, synthetic construct
  source 1..22: mol_type = other DNA
gtctcctctgacttcaacagcg

SEQ ID NO: 18 - DNA, 22 bp, synthetic construct
  source 1..22: mol_type = other DNA
accaccctgttgctgtagccaa

SEQ ID NO: 19 - DNA, 23 bp, synthetic construct
  source 1..23: mol_type = other DNA
gtgagccaaggaattttgtgttt

SEQ ID NO: 20 - DNA, 26 bp, synthetic construct
  source 1..26: mol_type = other DNA
ggtctgaactttgccattaatatctg

SEQ ID NO: 21 - DNA, 18 bp, synthetic construct
  source 1..18: mol_type = other DNA
ccatcttacaaggccttt

SEQ ID NO: 22 - DNA, 24 bp, synthetic construct
  source 1..24: mol_type = other DNA
tcaaaatagaagccagtggatgag

SEQ ID NO: 23 - DNA, 21 bp, synthetic construct
  source 1..21: mol_type = other DNA
aatggcatgcacaagaacatg

SEQ ID NO: 24 - DNA, 15 bp, synthetic construct
  source 1..15: mol_type = other DNA
cctgaggcaccacct

SEQ ID NO: 25 - DNA, 60 bp, Homo sapiens
  source 1..60: mol_type = other DNA
ctgtacctgatgcactggccttatgcctttgagtaaaagcagctgttaagtatgccctta

SEQ ID NO: 26 - DNA, 60 bp, Homo sapiens
  source 1..60: mol_type = other DNA
gatctttcagccacaaatgtcctggatggcagcagtatacagacaacctgtcacataata

SEQ ID NO: 27 - DNA, 60 bp, Homo sapiens
  source 1..60: mol_type = other DNA
cgccacagaaattaggaatctgaaataaacaggaaagcactatgtcttcaatgtggtctg

SEQ ID NO: 28 - DNA, 60 bp, Homo sapiens
  source 1..60: mol_type = other DNA
ccagactgtgagccaaggaattttgtgtttcttgccatcttacaaggccttttcagatat

SEQ ID NO: 29 - DNA, 60 bp, Homo sapiens
  source 1..60: mol_type = other DNA
ctcagaaaatgagaagatgggaatcagagtggatcagcttcaggtggggtggaagacaac

SEQ ID NO: 30 - DNA, 60 bp, Homo sapiens
  source 1..60: mol_type = other DNA
acgatggagaagaatatgatgtggaagcacatgaagaatcaaaagaacctgttgctgatg

SEQ ID NO: 31 - DNA, 60 bp, Homo sapiens
  source 1..60: mol_type = other DNA
tcctgaaagaagattagtaattattttgccaaagaaacataagaagaaaaaggagcggaa

SEQ ID NO: 32 - DNA, 60 bp, Homo sapiens
  source 1..60: mol_type = other DNA
cagacggaggaccatgatgggaacaagatcctcctacaggtgagctttccgttgaagagg

SEQ ID NO: 33 - DNA, 60 bp, Homo sapiens
  source 1..60: mol_type = other DNA
aaggatacacaggtgatggcttcacttgtacagatcccatatgtggtaaagggtactcaa

SEQ ID NO: 34 - DNA, 60 bp, Homo sapiens
  source 1..60: mol_type = other DNA
aatatcagcagactagagcagattcttccaaatgcacagcttgtgttcaggtgtgtcacg

SEQ ID NO: 35 - DNA, 60 bp, Homo sapiens
  source 1..60: mol_type = other DNA
tgctggagcaattgttgtgacttcactaataaagatgtgttcagagcaacattgttttgt

SEQ ID NO: 36 - DNA, 60 bp, Homo sapiens
  source 1..60: mol_type = other DNA
aaatgtcatcagcttctgaggaatctgtgctttctgtcccagaactccaggccagtctgc

SEQ ID NO: 37 - DNA, 60 bp, Homo sapiens
  source 1..60: mol_type = other DNA
aataacccagactaggaatgccagcatctgccaagcagatttgacgggatctgaactata

SEQ ID NO: 38 - DNA, 60 bp, Homo sapiens
  source 1..60: mol_type = other DNA
gctgcaaccctggatattctaacaggtttgtcaattaacagatgaacagatccatcacct

SEQ ID NO: 39 - DNA, 60 bp, Homo sapiens
  source 1..60: mol_type = other DNA
accagagcttaaaagctgcaaccctggatattctaacagcaagctaccgactagaaatac

SEQ ID NO: 40 - DNA, 60 bp, Homo sapiens
  source 1..60: mol_type = other DNA
tgctaacaaaaacagaacttttttagaaaatatgcaccaagtgagaatgggccaaatggg

SEQ ID NO: 41 - DNA, 60 bp, Homo sapiens
  source 1..60: mol_type = other DNA
aatagaagagtcacagagaaaaatgaacgcaaaagcggcgccaggaaattgaacaaaaac

SEQ ID NO: 42 - DNA, 60 bp, Homo sapiens
  source 1..60: mol_type = other DNA
atttaggaggattcgatggcaattttttgtggaacagaattggagcagttttgacgaagt

SEQ ID NO: 43 - DNA, 60 bp, Homo sapiens
  source 1..60: mol_type = other DNA
gagacaagcaaatctgacaggatggcatctgatgactttgacatagtgattgaggccatg

SEQ ID NO: 44 - DNA, 60 bp, Homo sapiens
  source 1..60: mol_type = other DNA
tgaaggagaagtttagcatgagaagtggaaagagaaatcaatccaagattaattaaaaga

SEQ ID NO: 45 - DNA, 60 bp, Homo sapiens
  source 1..60: mol_type = other DNA
agacatcagctacttaagcgccacgagaagagtaaaaagcgacaatatgaccaggaaatt

SEQ ID NO: 46 - DNA, 60 bp, Homo sapiens
  source 1..60: mol_type = other DNA
aaagcaggtcttttctacacagggagaacattttgtgaattcctgggtccagagagaatt

SEQ ID NO: 47 - DNA, 60 bp, Homo sapiens
  source 1..60: mol_type = other DNA
cctgatgaatggtggagagttacgcagataatgctaagtttgcaacagagagcacagaaa

SEQ ID NO: 48 - DNA, 60 bp, Homo sapiens
  source 1..60: mol_type = other DNA
catacaacacacagggagtgtcacattcaaggcctaacactgaggacatgtcactgtgtc

SEQ ID NO: 49 - DNA, 60 bp, Homo sapiens
  source 1..60: mol_type = other DNA
tcagggaaccctgatttactcctgtaactttccatttcacaaagggatttgtggaactga

SEQ ID NO: 50 - DNA, 60 bp, Homo sapiens
  source 1..60: mol_type = other DNA
gacaaggagcgctcggtgcgcatgatgctggaggagcagccttgccaacattccactaac

SEQ ID NO: 51 - DNA, 60 bp, Homo sapiens
  source 1..60: mol_type = other DNA
gtcacagcagagaagaaagagtgaatatgaaggttcccagagatatggatgatgccaagg

SEQ ID NO: 52 - DNA, 60 bp, Homo sapiens
  source 1..60: mol_type = other DNA
gataaaagtcctgaaacagttttgcagtcgaggagtttaataaagaaactgggcagcccc

SEQ ID NO: 53 - DNA, 60 bp, Homo sapiens
  source 1..60: mol_type = other DNA
ctttgaatctttgcatagaggtcaagataactccaggaggaaaagtagggatgggtcagc

SEQ ID NO: 54 - DNA, 60 bp, Homo sapiens
  source 1..60: mol_type = other DNA
tgtgcaaatccggaatgtcaatgtggagctttgtgcaaatgacatattagatggtgttga

SEQ ID NO: 55 - DNA, 60 bp, Homo sapiens
  source 1..60: mol_type = other DNA
tggctgcttaaactgctgctgtatttaaacttgaaacagatagaagtatatggcccttga

SEQ ID NO: 56 - DNA, 60 bp, Homo sapiens
  source 1..60: mol_type = other DNA
ttttgtggaatattcattgtactgaatattacccttgtgcttgccgagaagctcgcagag